(19) United States
(12) Patent Application Publication
(10) Pub. No.: US 2019/0083509 A1
(43) Pub. Date: Mar. 21, 2019

(54) OXYGENATED CHOLESTEROL SULFATES FOR THERAPY OF DISORDERS CAUSED BY AT LEAST ONE OF ATTENUATED LEPTIN ACTIVITY AND A LIPID STORAGE DISORDER

(71) Applicant: VIRGINIA COMMONWEALTH UNIVERSITY, Richmond, VA (US)

(72) Inventors: Shunlin REN, Richmond, VA (US); Jin Koung KIM, Richmond, VA (US)

(21) Appl. No.: 16/108,286

(22) Filed: Aug. 22, 2018

Related U.S. Application Data
(63) Continuation of application No. 15/517,395, filed on Apr. 6, 2017, now abandoned.
(60) Provisional application No. 62/062,260, filed on Oct. 10, 2014.

(51) Int. Cl. A61K 31/56; A61K 31/575; A61K 31/58

(52) U.S. Cl. CPC A61K 31/56; G01N 2800/044; A61K 31/58; A61K 31/575

(57) ABSTRACT

Methods and compositions are provided for the prevention and/or treatment of symptoms associated with lipid accumulation disorders caused by attenuated leptin activity and by lipid storage disorders. The methods involve administering at least one oxygenated cholesterol sulfate (OCS) to a subject with the disorder. The disorder may be acquired or congenital (hereditary).

### BACKGROUND OF THE INVENTION

### Field of the Invention

The present disclosure generally relates to the treatment of diseases and conditions associated with abnormal lipid accumulation. For instance, the present disclosure provides methods of treating individuals suffering from attenuated leptin activity (e.g. leptin deficiency and leptin resistance) and lipid storage disorders with at least one oxygenated cholesterol sulfate (OCS).

### BACKGROUND OF THE INVENTION

Stored fats (lipids), an important source of energy for the body, are constantly broken down and reassembled to balance the body's energy needs with the food available. However, in some congenital and acquired conditions, these processes go awry and harmful, unwanted lipid deposition occurs. Over time, abnormal lipid accumulation is harmful to many organs of the body, and can be life threatening.

The hormone leptin plays a key role in regulating several aspects of energy intake and expenditure, including appetite and hunger, metabolism, and food-seeking behavior. Leptin functions by binding to and activating the long form of its receptor (LEPR-B), which is found on the surface of cells in many organs and tissues of the body, including the hypothalamus. Leptin activity enables the central nervous system (CNS) to sense energy stores and as such is essential for the maintenance of normal energy homeostasis. For example, in healthy individuals, after food intake, leptin activity triggers a series of chemical signals that produce a feeling of fullness (satiety) and increase energy expenditure. However, in individuals with insufficient leptin activity, the chemical signaling is impaired.

A deficiency in leptin activity can be caused by several different factors. For example, some genetic abnormalities cause no or low levels of leptin, or defective leptin, to be produced by an individual (referred to herein as “leptin deficiency” or “LD”). A deficiency in leptin activity may also result from cells being or becoming resistant to leptin, so-called “leptin resistance” (LR). LR is generally defined as the failure of endogenous or exogenous leptin to promote anticipated salutary metabolic outcomes in states of over-nutrition or obesity. In leptin resistance, cells and/or tissue are unable to propagate leptin signaling in response to contact of the cells and/or tissue with leptin. Thus, adequate or even elevated levels of circulating leptin may be present, but an LR individual still experiences a deficiency in leptin activity. LR is caused by a number of factors, including genetic predispositions (e.g. a leptin receptor deficiency caused by mutations in the LEPR gene), obesity, lifestyle, etc. and combinations of these.

Deficiencies in leptin activity are associated with several untoward medical conditions, such as elevated levels of serum triglycerides and lipid accumulation in the liver, both of which can lead to high levels of impairment and mortality.

There remains a need for improved agents and methods for preventing, treating or reversing the effects of deficiencies in leptin activity, and diseases associated therewith.

Lipid storage disorders (or lipidoses) are a group of inherited metabolic disorders in which harmful amounts of lipids (fats) accumulate in some of the body's cells and tissues. People with these disorders either do not produce enough of one of the enzymes needed to metabolize lipids or they produce enzymes that do not work properly. Over time, this excessive storage of fats can cause extensive permanent cellular and tissue damage, particularly in the brain, peripheral nervous system, liver, spleen and bone marrow.

Unfortunately, these diseases can be fatal and the treatment options are non-existent or extremely limited. Treatment options are currently available for only two lipid storage diseases: Fabry disease and Gaucher's disease. Both diseases are caused by a missing or defective enzyme (alpha-galactosidase A for Fabry disease and glucocerebrosidase for Gaucher's disease). Both diseases are treated by enzyme replacement infusion therapy. Unfortunately, such therapy is relatively invasive and incidences of life-threatening severe allergic (anaphylactic) reactions have been reported for both. Very recently, an oral dosage form of glucocerebrosidase has become available to treat Gaucher's disease. However, there are still unanswered questions concerning long term efficacy of this treatment, and room for improvement exists.

There is a true unmet need in the treatment of attenuated leptin activity and lipid storage diseases.

### SUMMARY OF THE INVENTION

The present disclosure provides methods for preventing, reversing or treating symptoms of and/or conditions/diseases associated with lipid accumulation disorders, including those characterized by attenuated leptin activity (such as leptin-deficiency associated lipid accumulation (LDLA)), and leptin-resistance associated lipid accumulation (LRLA)), and lipid storage disorders (LSDs). The methods involve administering to a subject a therapeutically effective amount of an oxygenated cholesterol sulfate, the amount being sufficient to prevent, reverse or treat at least one symptom of the lipid accumulation disorder (e.g. LDLA, LRLA or an LSD). Such symptoms include but are not limited to hyperlipidemia, fatty liver diseases, and abnormal lipid accumulation in cells and tissues such as central and peripheral nervous tissue.

Provided herein are methods of treating leptin-deficiency associated lipid accumulation in a subject in need thereof. The methods comprise elevating in the subject an amount of at least one OCS such as 5-cholesten, 3-, 25-diol, 3-sulfate (25HC3S) of formula

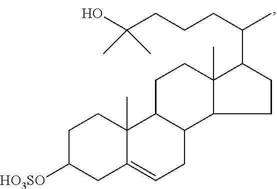

or a pharmaceutically acceptable salt thereof.

Also provided are substances which comprise at least one OCS, such as 5-cholesten, 3-, 25-diol, 3-sulfate (25HC3S), or such as or pharmaceutically acceptable salts thereof, for use in methods for treating leptin-deficiency associated lipid accumulation in a subject, as are substances which comprise at least one OCS such as 25-hydroxycholesterol (25HC), or a pharmaceutically acceptable salt thereof, for use in methods for treating leptin-deficiency associated lipid accumulation in a subject.

Also provided are substances for use in methods for treating lipid accumulation associated disorders with attenuated leptin activity or a lipid storage disorder in a subject, in, for example, organs such as the liver or in tissue of the nervous system. The substances comprising one or both of: i) a nucleic acid encoding, and capable of producing, an enzyme in the subject, which enzyme is capable of promoting the production of at least on endogenous OCS such as 25HC3S in the subject; and ii) an enzyme capable of promoting the production of at least one endogenous OCS such as 25HC3S in the subject.

Further provided is the use of at least one OCS such as 5-cholesten, 3-, 25-diol, 3-sulfate (25HC3S) or a pharmaceutically acceptable salt thereof, in the manufacture of a medicament for use in a method for treating lipid accumulation associated disorders with attenuated leptin activity or a lipid storage disorder in a subject.

Also provided is the use of at least one OCS such as 25-hydroxycholesterol (25HC), or a pharmaceutically acceptable salt thereof, in the manufacture of a medicament for use in methods for treating leptin-deficiency associated lipid accumulation in a subject, e.g., lipid accumulation in the liver of the subject.

Further provided is one or both of: i) a nucleic acid encoding, and capable of producing, an enzyme in the subject, which enzyme is capable of promoting the production of at least one endogenous OCS such as 25HC3S in the subject; and ii) an enzyme capable of promoting the production of at least one endogenous OCS such as 25HC3S in the subject; in the manufacture of a medicament for use in a method for treating leptin-deficiency associated lipid accumulation in a subject, e.g., lipid accumulation in the liver of the subject.

Further aspects of the disclosure include:
1. A method of treating at least one of i) attenuated leptin activity and ii) lipid storage disorder in a subject in need thereof,
   the method comprising elevating in the subject an amount of at least one oxygenated cholesterol sulfate (OCS), or a pharmaceutically acceptable salt thereof, wherein the elevating is sufficient to treat or alleviate symptoms of at least one of attenuated leptin activity and lipid storage disorder.
2. The method of 1, wherein the at least one OCS is selected from 5-cholesten-3, 25-diol, 3-sulfate (25HC3S); 5-cholesten, 3b, 25-diol, disulfate (25HCDS); (5-cholestene, 3, 27-diol, 3-sulfate); (5-cholestene, 3, 27-diol, 3, 27-disulfate); (5-cholestene, 3,7-diol, 3-sulfate); (5-cholestene, 3,7-diol, 3,7-disulfate); (5-cholestene, 3, 24-diol, 3-sulfate); (5-cholestene, 3, 24-diol, 3, 24-disulfate); and (5-cholestene, 3-ol, 24, 25-epoxy 3-sulfate).
3. The method of 1, wherein the elevating results in the subject having reductions of lipids and/or lipid deposits in one or more of the circulatory system, blood or internal organs of the subject.
4. The method of 1, wherein the elevating results in a reduction of lipids and/or lipid deposits in the liver of the subject.
5. The method of 1, wherein the elevating comprises administering the at least one OCS or a pharmaceutically acceptable salt thereof to the subject.
6. The method of 5, wherein the at least one OCS or a pharmaceutically acceptable salt thereof is administered in an amount ranging from 0.1 mg/kg to 100 mg/kg, based on the body mass of the subject.
7. The method of 6, wherein the at least one OCS or a pharmaceutically acceptable salt thereof is administered in an amount ranging from 10 mg/kg to 30 mg/kg, based on the body mass of the subject.
8. The method of any one of 5 to 7 wherein the administering comprises at least one of oral administration, enteric administration, sublingual administration, transdermal administration, intravenous administration, peritoneal administration, parenteral administration, administration by injection, subcutaneous injection, and intramuscular injection.
9. The method of 1, wherein the elevating comprises promoting endogenous production of the at least one OCS in the subject.
10. The method of encoding, and capable of producing, an enzyme in the subject, which enzyme is capable of promoting endogenous production of the at least one OCS in the subject; and
   an enzyme capable of promoting endogenous production of the at least one OCS in the subject.
11. The method of 10, wherein the at least one OCS is 25HC3S and the enzyme is SULT2B1b.
12. The method of 10 or 11, which further comprises administering to the subject a substrate of the enzyme, which substrate is capable of endogenous production of the at least one OCS in the subject.
13. The method of 12, wherein the substrate is 25-hydroxycholesterol (25HC) or a pharmaceutically acceptable salt thereof.
14. The method of any one of the preceding, which comprises treating one or more symptoms of lipid accumulation caused by attenuated leptin activity.
15. The method of 14, wherein the attenuated leptin activity is, or is caused by, leptin deficiency or by leptin resistance.
16. The method of any one of the preceding, which comprises decreasing one or both of serum triglycerides and hepatic neutral lipids in the subject.
17. The method of 16, wherein the serum triglycerides are lowered by at least 20%.
18. The method of 16 or 17, wherein the hepatic neutral lipids are lowered by at least 20%.
19. The method of any one of the preceding, wherein the lipid accumulation caused by attenuated leptin activity is the result of obesity or a genetic predisposition.
20. The method of any one of the preceding, wherein the lipid accumulation caused by attenuated leptin activity is leptin resistance caused by a mutation in a gene encoding a leptin receptor in the subject.
21. The method of 20, wherein the subject has impaired binding of leptin to a leptin receptor due to the mutation.
22. The method of any one of the preceding, further comprising
   prior to the elevating, measuring a value of one or more indicia of attenuated leptin activity in the subject;
   comparing the value with at least one negative control value or negative control range for the one or more indicia from a control population of individuals who do not have attenuated leptin activity; and
   if the value differs from that of the negative control value or falls outside the negative control range, then
   concluding that the subject has attenuated leptin activity.
23. The method of any one of the preceding, further comprising
   prior to the elevating, measuring a value of one or more indicia of attenuated leptin activity in the subject;
   comparing the value to at least one positive control value or positive control range for the one or more indicia from a control population of individuals who have attenuated leptin activity; and
   if the value is the same as that of the positive control value or falls within the positive control range, then
   concluding that the subject has attenuated leptin activity.
24. The method of 22 or 23, wherein the one or more indicia of attenuated leptin activity is selected from serum leptin levels, body mass index, and serum triglyceride levels.
25. The method of 22 or 23, further comprising
   after the elevating, monitoring the one or more symptoms of attenuated leptin activity in the subject.
26. A substance which comprises at least one oxygenated cholesterol sulfate (OCS) or a pharmaceutically acceptable salt thereof, for use in a method for treating at least one of i) attenuated leptin activity and ii) lipid storage disorder.
27. The substance of 26, wherein the at least one OCS is selected from 5-cholesten-3, 25-diol, 3-sulfate (25HC3S); 5-cholesten, 3b, 25-diol, disulfate (25HCDS); (5-cholestene, 3, 27-diol, 3-sulfate); (5-cholestene, 3, 27-diol, 3, 27-disulfate); (5-cholestene, 3,7-diol, 3-sulfate); (5-cholestene, 3,7-diol, 3,7-disulfate); (5-cholestene, 3, 24-diol, 3-sulfate); (5-cholestene, 3, 24-diol, 3, 24-disulfate); and (5-cholestene, 3-ol, 24, 25-epoxy 3-sulfate).
28. The substance for use of 26, wherein the method comprises administering to the subject the at least one OCS or a pharmaceutically acceptable salt thereof in an amount ranging from 0.01 mg/kg to 100 mg/kg, based on the body mass of the subject.
29. The substance for use of 28, wherein the method comprises administering to the subject the at least one OCS or a pharmaceutically acceptable salt thereof in an amount ranging from 10 mg/kg to 30 mg/kg, based on the body mass of the subject.
30. The substance for use of any one of 26 to 29, wherein the method comprises at least one of oral administration, enteric administration, sublingual administration, transdermal administration, intravenous administration, peritoneal administration, parenteral administration, administration by injection, subcutaneous injection, and intramuscular injection.
31. The substance of any one of 26 to 30, wherein the attenuated leptin activity is, or is caused by, leptin deficiency or leptin-resistance.
32. A substance which comprises 25-hydroxycholesterol (25HC) or a pharmaceutically acceptable salt thereof, for use in a method for treating at least one of i) attenuated leptin activity and ii) lipid storage disorder.
33. The substance for use of 32, wherein the method comprises the conversion, in the body of the subject, of the 25-hydroxycholesterol, or pharmaceutically acceptable salt thereof, into 25HC3S or a pharmaceutically acceptable salt thereof.
34. The substance of any one of 32 and 33, wherein the attenuated leptin activity is, or is caused by, leptin deficiency or leptin-resistance.
35. A substance for use in a method for treating at least one of i) attenuated leptin activity and ii) lipid storage disorder in a subject, the substance comprising one or both of:
   a nucleic acid encoding, and capable of producing, an enzyme in the subject, which enzyme is capable of promoting the endogenous production of at least one oxygenated cholesterol sulfate (OCS) in the subject; and
   an enzyme capable of promoting the endogenous production of at least one OCS in the subject.
36. The substance of 35, wherein the at least one OCS is selected from 5-cholesten-3, 25-diol.

3-sulfate (25HC3S); 5-cholesten, 3b, 25-diol, disulfate (25HCDS); (5-cholestene, 3, 27-diol, 3-sulfate); (5-cholestene, 3, 27-diol, 3, 27-disulfate); (5-cholestene, 3,7-diol, 3-sulfate); (5-cholestene, 3,7-diol, 3,7-disulfate); (5-cholestene, 3, 24-diol, 3-sulfate); (5-cholestene, 3, 24-diol, 3, 24-disulfate); and (5-cholestene, 3-ol, 24, 25-epoxy 3-sulfate).
37. The substance for use of 36, wherein the OCS is 25HC3S and the enzyme is SULT2B1b.
38. The substance for use of any one of 26 to 37, wherein the method comprises treating one or more symptoms of a lipid accumulation.
39. The substance for use of any one of 26 to 38, wherein the method comprises decreasing one or both of serum triglycerides and hepatic neutral lipids in the subject.
40. The substance for use of 39, wherein the serum triglycerides are lowered by at least 20%.

41. The substance for use of 39 or 40, wherein the hepatic neutral lipids are lowered by at least 35%.
42. The substance for use of any one of claims 27 to 40, wherein the attenuated leptin activity is the result of obesity or a genetic predisposition.
43. The substance for use of any one of 26 to 42, wherein the attenuated leptin activity is the result of a mutation in a gene encoding a leptin receptor in the subject. 44. The substance for use of 43, wherein the subject has impaired binding of leptin to a leptin receptor due to the mutation.
45. Use of an oxygenated cholesterol sulfate (OCS) or a pharmaceutically acceptable salt thereof, in the manufacture of a medicament for use in a method for treating at least one of i) attenuated leptin activity and ii) lipid storage disorder in a subject.
46. Use of 45, wherein the attenuated leptin activity is, or is caused by, leptin deficiency or leptin resistance.
47. Use of 25-hydroxycholesterol (25HC), or a pharmaceutically acceptable salt thereof, in the manufacture of a medicament for use in a method for treating at least one of i) attenuated leptin activity and ii) lipid storage disorder in a subject.
48. Use of 47, wherein the attenuated leptin activity is, or is caused by, leptin deficiency or leptin resistance.
49. Use of one or both of:
   a nucleic acid encoding, and capable of producing, an enzyme in the subject, which enzyme is capable of promoting the endogenous production of at least one OCS in the subject; and
      an enzyme capable of promoting the endogenous production of at least one OCS in the subject;
   in the manufacture of a medicament for use in a method for treating at least one of i) attenuated leptin activity and ii) lipid storage disorder in the subject.

50. Use of 49, wherein the attenuated leptin activity is, or is caused by, leptin deficiency or leptin resistance.
51. The method of 1, wherein said lipid storage disorder is selected from the group consisting of: Gaucher disease, Niemann-Pick disease, Fabry disease, Farber's disease, GM1 gangliosidosis, GM2 gangliosidosis, Krabbe disease, metachromatic leukodystrophy (MLD), and an acid lipase deficiency disorder.
52. The method of 51, wherein said GM2 gangliosidosis is Tay-Sachs disease or Sandhoff disease)
53. The method of 51, wherein said MLD is late infantile MLD, juvenile MLD or adult MLD.
54. The method of 51, wherein said acid lipase deficiency disorder is Wolman's disease or cholesteryl ester storage disease.
55. A method of treating leptin-deficiency associated lipid accumulation in a subject in need thereof,
   the method comprising elevating in said subject the amount of 5-cholesten, 3-, 25-diol, 3-sulfate (25HC3S) of formula

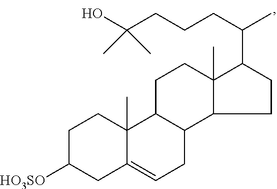

or a pharmaceutically acceptable salt thereof.
56. The method of 55, wherein said elevating in said subject the amount of 25HC3S or a pharmaceutically acceptable salt thereof comprises administering 25HC3S or a pharmaceutically acceptable salt thereof to said subject.
57. The method of 56, wherein the 25HC3S or a pharmaceutically acceptable salt thereof is administered in an amount ranging from 0.1 mg/kg to 100 mg/kg, based on the body mass of said subject.
58. The method of 57, wherein the 25HC3S or a pharmaceutically acceptable salt thereof is administered in an amount ranging from 10 mg/kg to 30 mg/kg, based on the body mass of said subject.
59. The method of any one of 56 to 58 wherein the administering comprises at least one of oral administration, enteric administration, sublingual administration, transdermal administration, intravenous administration, peritoneal administration, parenteral administration, administration by injection, subcutaneous injection, and intramuscular injection.
60. The method of 55, wherein said elevating in said subject the amount of 25HC3S or a pharmaceutically acceptable salt thereof comprises administering 25-hydroxycholesterol (25HC) or a pharmaceutically acceptable salt thereof to said subject.
61. The method of 60, wherein the 25-hydroxycholesterol or a pharmaceutically acceptable salt thereof is administered in an amount ranging from 0.1 mg/kg to 100 mg/kg, based on the body mass of said subject.
62. The method of 61, wherein the 25-hydroxycholesterol or a pharmaceutically acceptable salt thereof is administered in an amount ranging from 10 mg/kg to 30 mg/kg, based on the body mass of said subject.
63. The method of any one of 60 to 62 wherein the administering comprises at least one of oral administration, enteric administration, sublingual administration, transdermal administration, intravenous administration, peritoneal administration, parenteral administration, administration by injection, subcutaneous injection, and intramuscular injection.
64. The method of 55, wherein said elevating in said subject the amount of 25HC3S or a pharmaceutically acceptable salt thereof comprises promoting the production of endogenous 25HC3S in said subject.
65. The method of 64, which comprises administering to said subject one or both of:
   a nucleic acid encoding, and capable of producing, an enzyme in the subject, which enzyme is capable of promoting the production of endogenous 25HC3S in said subject; and
   an enzyme capable of promoting the production of endogenous 25HC3S in said subject.
66. The method of 64, wherein the enzyme is SULT2B1b.
67. The method of 65 or 66, which further comprises administering to said subject a substrate of the enzyme, which substrate is capable of producing endogenous 25HC3S in said subject.
68. The method of 67, wherein the substrate is 25-hydroxycholesterol (25HC) or a pharmaceutically acceptable salt thereof.
69. The method of any one of the preceding, which comprises treating one or more symptoms of leptin-deficiency associated lipid accumulation.
70. The method of any one of the preceding, which comprises decreasing one or both of serum triglycerides and hepatic neutral lipids in said subject.
71. The method of claim 70, wherein said serum triglycerides are lowered by at least 20%.
72. The method of claim 70 or 71, wherein said hepatic neutral lipids are lowered by at least 20%.
73. The method of any one of the preceding, wherein said leptin-deficiency associated lipid accumulation is the result of obesity or a genetic predisposition to leptin deficiency.
74. The method of any one of the preceding, wherein said leptin-deficiency associated lipid accumulation is the result of a mutation in a gene encoding a leptin receptor in said subject.
75. The method of claim 74, wherein the subject has impaired binding of leptin to a leptin receptor due to said mutation.
76. The method of any one of the preceding, further comprising
   prior to said elevating, measuring a value of one or more indicia of leptin deficiency in said subject;
   comparing said value with at least one negative control value or negative control range for said one or more indicia from a control population of individuals who are not leptin deficient; and
   if said value differs from that of said negative control value or falls outside said negative control range, then
   concluding that said subject has leptin deficiency.
77. The method of any one of the preceding, further comprising
   prior to said elevating, measuring a value of one or more indicia of leptin deficiency in said subject;
   comparing said value to at least one positive control value or positive control range for said one or more indicia from a control population of individuals who are leptin deficient; and
   if said value is the same as that of said positive control value or falls within said positive control range, then
   concluding that said subject has leptin deficiency.
78. The method of 76 or 77, wherein said one or more indicia of leptin deficiency is selected from serum leptin levels, body mass index, and serum triglyceride levels.
79. The method of 69 or 70, further comprising after said elevating, monitoring said one or more symptoms of leptin-deficiency associated lipid accumulation in said subject.
80. The method of any one of 55 to 79, wherein the leptin-deficiency associated lipid accumulation comprises leptin-resistance associated lipid accumulation.
81. A substance which comprises 5-cholesten, 3-, 25-diol, 3-sulfate (25HC3S) of formula

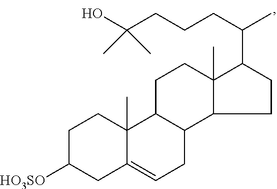

or a pharmaceutically acceptable salt thereof, for use in a method for treating leptin-deficiency associated lipid accumulation in a subject.
82. The substance for use of 81, wherein the method comprises administering to said subject 25HC3S or a pharmaceutically acceptable salt thereof in an amount ranging from 0.01 mg/kg to 100 mg/kg, based on the body mass of said subject
83. The substance for use of 82, wherein the method comprises administering to said subject 25HC3S or a pharmaceutically acceptable salt thereof in an amount ranging from 10 mg/kg to 30 mg/kg, based on the body mass of said subject.
84. The substance for use of any one of claims 80 to 83, wherein the method comprises at least one of oral administration, enteric administration, sublingual administration, transdermal administration, intravenous administration, peritoneal administration, parenteral administration, administration by injection, subcutaneous injection, and intramuscular injection.
85. The substance of any one of 81 to 84, wherein the leptin-deficiency associated lipid accumulation comprises leptin-resistance associated lipid accumulation.
86. A substance which comprises 25-hydroxycholesterol (25HC), or a pharmaceutically acceptable salt thereof, for use in a method for treating leptin-deficiency associated lipid accumulation in a subject.
87. The substance for use of 86, wherein the method comprises the conversion, in the body of the subject, of said 25-hydroxycholesterol, or pharmaceutically acceptable salt thereof, into 25HC3S or a pharmaceutically acceptable salt thereof.
88. The substance of any one of 86 and 87, wherein the leptin-deficiency associated lipid accumulation comprises leptin-resistance associated lipid accumulation.
89. A substance for use in a method for treating leptin-deficiency associated lipid accumulation in a subject, the substance comprising one or both of:
   a nucleic acid encoding, and capable of producing, an enzyme in the subject, which enzyme is capable of promoting the production of endogenous 25HC3S in said subject; and
   an enzyme capable of promoting the production of endogenous 25HC3S in said subject.
90. The substance for use of 89, wherein the enzyme is SULT2B 1 b.
91. The substance for use of any one of 81 to 90, wherein the method comprises treating one or more symptoms of leptin-deficiency associated lipid accumulation.
92. The substance for use of any one of 81 to 91, wherein the method comprises decreasing one or both of serum triglycerides and hepatic neutral lipids in said subject.
93. The substance for use of 92, wherein said serum triglycerides are lowered by at least 20%.
94. The substance for use of 92 or 93, wherein said hepatic neutral lipids are lowered by at least 35%.

95. The substance for use of any one of 81 to 94, wherein said leptin-deficiency associated lipid accumulation is the result of obesity or a genetic predisposition to leptin deficiency
96. The substance for use of any one of 81 to 95, wherein said leptin-deficiency associated lipid accumulation is the result of a mutation in a gene encoding a leptin receptor in said subject.
97. The substance for use of 96, wherein the subject has impaired binding of leptin to a leptin receptor due to said mutation.
98. Use of 5-cholesten, 3-, 25-diol, 3-sulfate (25HC3S) of formula

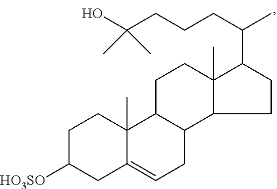

or a pharmaceutically acceptable salt thereof, in the manufacture of a medicament for use in a method for treating leptin-deficiency associated lipid accumulation in a subject.
99. Use of 98, wherein the leptin-deficiency associated lipid accumulation comprises leptin-resistance associated lipid accumulation.
100. Use of 25-hydroxycholesterol (25HC), or a pharmaceutically acceptable salt thereof, in the manufacture of a medicament for use in a method for treating leptin-deficiency associated lipid accumulation in a subject.
101. Use of 100, wherein the leptin-deficiency associated lipid accumulation comprises leptin-resistance associated lipid accumulation.
102. Use of one or both of:
   a nucleic acid encoding, and capable of producing, an enzyme in the subject, which enzyme is capable of promoting the production of endogenous 25HC3S in said subject; and
   an enzyme capable of promoting the production of endogenous 25HC3S in said subject;
      in the manufacture of a medicament for use in a method for treating leptin-deficiency associated lipid accumulation in a subject.
103. Use of 102, wherein the leptin-deficiency associated lipid accumulation comprises leptin-resistance associated lipid accumulation.

Other features and advantages of the present invention will be set forth in the description of invention that follows, and in part will be apparent from the description or may be learned by practice of the invention. The invention will be realized and attained by the compositions and methods particularly pointed out in the written description and claims hereof.

### BRIEF DESCRIPTION OF THE DRAWINGS

The present invention is further described in the description of invention that follows, in reference to the noted plurality of non-limiting drawings, wherein:

FIG. 1A-D. Serum lipid levels of male Zucker Leptin-Deficient (obese fa/fa) animals after 21 days of treatment with 25HC3S at the indicated levels. A, HDL cholesterol; B, LDL cholesterol; C, total serum cholesterol; D, triglycerides. All the values are expressed as mean±SD. *p<0.05 and **p<0.01 versus vehicle-treated animals.

FIG. 2A-D. Hepatic lipid levels of male Zucker Leptin-Deficient (obese fa/fa) animals after 21 days of treatment with 25HC3S at the indicated levels. A, triglycerides; B, total cholesterol; C, free fatty acids; D, free cholesterol. Each individual was normalized by liver weight all the values are expressed as mean±SD. *p<0.05 and **p <0.01 versus vehicle-treated animals.

FIG. 3A-E. Liver morphology studies. Hematoxylin and eosin (HE) stained liver sections from animals treated with A, vehicle; B, 10 mg/kg/day 25HC3S PO; C, 30 mg/kg/day 25HC3S PO; D, 100 mg/kg PO; E, 50 mg/kg IP.

FIG. 4A-E. Liver morphology studies. Oil Red O stained liver sections from animals treated with A, vehicle; B, 10 mg/kg/day 25HC3S PO; C, 30 mg/kg/day 25HC3S PO; D, 100 mg/kg PO; E, 50 mg/kg IP.

### DETAILED DESCRIPTION

The present disclosure provides methods for the treatment of disorders characterized by abnormal lipid accumulation (LA). The methods are based on the surprising discovery that administration of an OCS to mammals which have existing abnormal, harmful deposits of lipids (e.g. lipid globules in liver or other organs or tissues wherein deposition is inappropriate), results in a decrease or elimination of the lipid deposits and the prevention of additional lipid accumulation. Thus, administration of the OCS prevents abnormal lipid deposition and reverses lipid deposition (accumulation) that is extant when treatment begins. Disorders that are so-treated are referred to herein by phrases such as “lipid accumulation disorders”, “lipid deposition disorders”, etc. and include but are not limited to:
I. disorders which result from a lack or attenuation of leptin activity, due to, for example,
   i) a genetic mutation that causes low levels of leptin production, or production of a non- or poorly functioning leptin molecule, such as occurs in leptin deficiency (LD); or
   ii) a defect in leptin signaling, caused by e.g. a congenital or acquired abnormality or deficiency in the functioning of the leptin receptor, e.g. due to a genetic mutation of the leptin receptor, or due to an acquired loss of receptor sensitivity to leptin binding such as that which occurs in leptin resistance (LR); and
II. lipid storage disorders, which are generally congenital.

The term “attenuated leptin activity” as used herein thus embraces leptin deficiency (LD) and leptin resistance (LR) as characterized in i) and ii) above. Similarly, the term “leptin-deficiency associated lipid accumulation” as used herein embraces lipid accumulation associated with leptin deficiency (LD) and leptin resistance (LR), as characterized in i) and ii) above.

The methods involve administering a therapeutically effective amount of at least one oxygenated cholesterol sulfate (OCS) to the subject. Proper lipid homeostasis is thus maintained or restored and the effects of the disease (e.g. lipid accumulation, LA, are prevented, reversed, attenuated or eradicated. As used herein, “treatment” or “treating” encompasses therapeutic treatment of existing disease, prophylactic administration to prevent or to prevent recurrence of disease, as well as managing disease.

Considering that there remains a need for improved agents and methods for treating the effects of lipid accumulation disorders such as LDLA, LRLA, and lipid storage disorders, the results of the present disclosure are surprising.

Examples of OCSs that are used in the methods and compositions described herein include but are not limited to 5-cholesten-3, 25-diol, 3-sulfate (25HC3S); 5-cholesten, 3b, 25-diol, disulfate (25HCDS); (5-cholestene, 3, 27-diol, 3-sulfate); (5-cholestene, 3, 27-diol, 3, 27-disulfate); (5-cholestene, 3,7-diol, 3-sulfate); (5-cholestene, 3,7-diol, 3,7-disulfate);

(5-cholestene, 3, 24-diol, 3-sulfate); (5-cholestene, 3, 24-diol, 3, 24-disulfate); and (5-cholestene, 3-ol, 24, 25-epoxy 3-sulfate). Disclosure of 25HC3S is found in, e.g., U.S. Pat. No. 8,399,441, which is incorporated herein by reference in its entirety. Disclosure of 25HCDS is found, e.g., in WO 2013/154752, which is incorporated by reference in its entirety. In certain aspects, the OCS are selected from 5-cholesten-3, 25-diol, 3-sulfate (25HC3S) and 5-cholesten, 3b, 25-diol, disulfate (25HCDS) (either alone or in combination). In further aspects, the OCS is 5-cholesten-3, 25-diol, 3-sulfate (25HC3S).

By “25-hydroxycholesterol-3-sulfate (25HC3S)” we mean a compound of the structure:

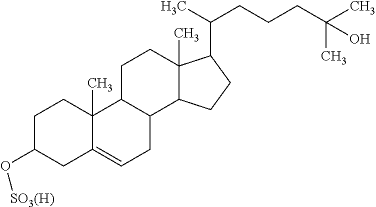

25HC3S is described, for example, in published United States patent application US-2010-0273761 (Ren et al.), the complete content of which is herein incorporated by reference in its entirety. US-2010-0273761 does not, however, teach that 25HC3S may be efficacious for the treatment, prevention or management of disorders which result from a lack or attenuation of leptin activity or lipid storage disorders in patients.

By “5-cholesten, 3b, 25-diol, disulfate (25HCDS)” we mean a compound of the structure

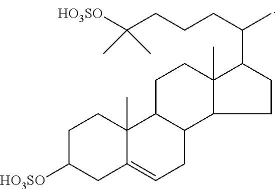

25HCDS is described, for example, in published United States patent application US-20150072962 (Ren et al.), the complete content of which is herein incorporated by reference in its entirety. US-20150072962 does not, however, teach that 25HC3S may be efficacious for the treatment, prevention or management of disorders which result from a lack or attenuation of leptin activity or lipid storage disorders in patients.

The OCS (e.g.25HC3S or 25HCDS) may be in the form of a pharmaceutically acceptable salt. The pharmaceutically acceptable salt may be a salt formed by the loss of the hydrogen atom on the sulfate group of the parent OCS molecule.

The pharmaceutically acceptable salt may, for example, be an alkali metal salt (e.g., a lithium, sodium or potassium salt), an alkaline earth metal salt (e.g., a calcium salt) or an ammonium salt. The pharmaceutically acceptable salt may, for example, be a sodium, potassium, calcium, lithium or ammonium salt.

One example of such a salt is a sodium salt of 25HC3S, for example a mono-addition sodium salt of 25HC3S, such as the mono-addition salt formed by loss of the hydrogen atom on the sulfate group of 25HC3S, i.e. the compound having the formula:

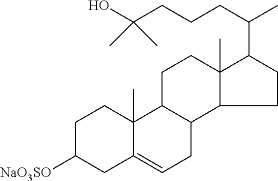

Another example of such a salt is a sodium salt of 25HCDS, for example a di-addition salt or a mono-addition salt. A di-addition salt is formed by loss of the hydrogen atoms on each of the two sulfate groups of the 25HCDS molecule. A mono-addition salt is formed by the loss of the hydrogen atom on only one of the two sulfate groups of the 25HCDS molecule (either at the 3β or the 25-position of the molecule), i.e. the compound having the formula:

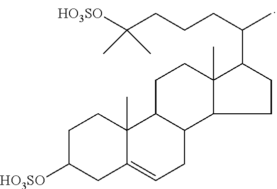

For the avoidance of doubt, it is emphasized that references throughout this specification to “25HC3S” or to “25HCDS” or to other OCSs include pharmaceutically acceptable salts unless explicitly indicated otherwise.

As used herein, the phrase “a substance which comprises” a specified compound includes within its scope both the compound itself (i.e., the substance “is” the specified compound) and a composition which comprises the specified compound together with other compounds. For example, a substance which comprises an OCS or a pharmaceutically acceptable salt thereof may be: (a) an OCS such as 25HC3S or 25HCDS, (b) a pharmaceutically acceptable salt of an OCS such as 25HC3S or 25HCDS, (c) a composition that comprises an OCS such as 25HC3S or 25HCDS and (d) a composition that comprises a pharmaceutically acceptable salt of an OCS such as 25HC3S or 25HCDS.

The amount of the at least one OCS to be administered may vary depending on characteristics of the subject to whom it is administered (for example, the species, gender, age, genetic makeup, general health, etc.). However, a clinically relevant amount (a therapeutically effective dose) will generally be in the range of from about 0.01 mg/kg to about 100 mg/kg, based on body mass of the subject. For example, the dose may range from about 0.1 mg/kg to about 50 mg/kg, or from about 1 to 30 mg/kg, or from about 3 to 20, including ranges of from about 0.5 to about 40, about 1 to about 10 mg/kg, about 10 to about 30 mg/kg, etc. For example, the dose may be about 0.01, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 20.5, 21.0, 21.5, 22.0, 22.5, 23.0, 23.5, 24.0, 24.5, 25.0, 25.5, 26.0, 26.5, 27.0, 27.5, 28.0, 28.5, 29.0, 29.5 or 30.0 mg/kg (including all decimal fractions in between each value at, e.g., 0.01 mg/kg intervals), or more e.g. about 30, 35, 40, 45, 50, 55, 60, 65, 70, 75, 80, 85, 90, 95 or 100 mg/kg (including all integers and decimal fractions in between each value at, e.g., 0.01 mg/kg intervals).

The timing or frequency of administration varies depending on, for example, the disease or disorder that is treated, the severity of the disease/disorder, the individual being treated (e.g. age, gender, overall health, presence or absence of other disease conditions and/or other treatments being pursued concomitantly, etc.). For example, administration may occur as frequently as about 1-4 times per day (e.g. 1, 2, 3, 4, or more times per day), or may be only once every other day, or once every 3-6 days, or once per week or 10 days, or once every 2 or 3 weeks, or once per month, or even less frequently, e.g. once every 2, 3, 4, or 5 months, or e.g. once or twice per year. Alternatively, or in addition, the dosing may be continuous, e.g. via IV administration.

Generally, more frequent or continuous dosing (and possibly higher quantities of OCS) is used when severe or acute symptoms are present, and/or soon after diagnosis to “load” the system with the OCS. In contrast, once symptoms have subsided to a desired level, a subject may be dosed less frequently, and/or the amount of OCS may be decreased, e.g. to a maintenance dosage. For example, the loading dose may range from 3 to 20 mg/kg per day for a period of 1 to 10 days, followed by a maintenance dose of 0.1 to 10 mg/kg per day.

Accordingly, administration generally begins as soon as a diagnosis is confirmed and continues until a desired salutary effect is observed, e.g. disappearance of fatty deposits. In some aspects, administration continues on an ongoing basis to prevent the recurrence of disease or is halted if the disease is considered to be controlled by other means, e.g. in some cases, a recurrence of LR is prevented by changes in diet (lower caloric intake, changes in quantity and type of fats in diet, etc.) and lifestyle (e.g. increased exercise). However, especially for congenital conditions which are due to genetic mutations, treatment is usually ongoing throughout the lifetime of an individual. Further, such individuals benefit from more frequent dosing and/or higher doses and/or dosing that occurs in a timed or slow release manner so that OCS administration is relatively constant.

In some aspects, a therapeutically effective dose of at least one OCS or a pharmaceutically acceptable salt thereof is administered directly to a subject in need thereof, e.g., a formulation or composition containing the agent(s) is administered directly to the subject to address the effects of LD, LR, LDLA, LRLA and/or lipid storage disorders in relevant portions of the body of the subject (e.g., circulatory system, blood, internal organs, liver, cells of the central and peripheral nervous system, etc.). In one aspect, the compositions include at least one substantially purified OCS, and a pharmacologically suitable (physiologically compatible) carrier. The preparation of compositions suitable for administration is known to those of skill in the art. Typically, such compositions are prepared either as liquid solutions or suspensions, however solid forms such as tablets, pills, powders and the like are also contemplated. Solid forms suitable for solution in, or suspension in, liquids prior to administration may also be prepared. The preparation may also be emulsified. The active ingredients may be mixed with excipients which are pharmaceutically acceptable and compatible with the active ingredients. Suitable excipients are, for example, water, saline, dextrose, glycerol, ethanol and the like, or combinations thereof. In addition, the composition may contain minor amounts of auxiliary substances such as wetting or emulsifying agents, pH buffering agents, and the like. Various thickeners, flavorings, diluents, emulsifiers, dispersing aids or binders and the like may be included in compositions for oral delivery. The composition of the present disclosure may contain any such additional ingredients so as to provide the composition in a form suitable for administration. The final amount of active agent in the formulations may vary. However, in general, the amount in the formulations will be from about 1-99%. 25HC3S may be synthesized, for example, as described in published U.S. patent applications 20070275939 and 20100273761 (Ren et al.), the complete contents of both of which are hereby incorporated by reference in their entireties.

Alternatively, or in addition, a precursor of an OCS such as 25HC3S is administered to the subject and the precursor is converted by the body, e.g. to 25HC3S. Exemplary 25HC3S precursors include but are not limited to 25-hydroxycholesterol (25HC). In all aspects, the level of biologically available OCS is increased (elevated, augmented, etc.) within the body, or within relevant portions of the body (circulatory system, blood, internal organs, liver, etc.), as a result of conversion of the precursor and the beneficial effects of elevated levels of OCS are brought about.

Other aspects of the disclosure include administering a nucleic acid encoding an enzyme that produces an OCS such as 25HC3S or 25HCDS, or is active in the biological synthesis pathway that produces an OCS such as 25HC3S or 25HCDS, in order to increase the level of biologically available OCS. Exemplary enzymes that may be overexpressed in a subject in need thereof in this manner include but are not limited to the hydroxysterol sulfotransferase enzyme SULT2B1b. Overexpressed SULT2B1b catalyzes sulfation of the naturally occurring endogenous substrate 25HC within the subject, converting it to 25HC3S, thereby increasing the concentration of 25HC3S in the subject. The administration of nucleic acids, e.g. in a suitable vector, is done using suitable methodology known in the art. This aspect of the present disclosure may also optionally include co-administration, together with the nucleic acid, of an exogenous precursor of an OCS such as 25HC3S (e.g. 25HC). The present disclosure also encompasses a treatment method in which both an OCS such as 25HC3S and a nucleic acid encoding a relevant enzyme such as SULT2B1b or an enzymatically active form thereof (with or without a substrate such as 25HC) are administered. Techniques and guidelines for such gene therapy are described, for example, in “Present and future of adeno associated virus based gene therapy approaches.” Recent Pat Endocr Metab Immune Drug Discov. 2012 January; 6(1), 47-66. “Gene Delivery System: A Developing Arena of Study for the New Era of Medicine” Recent Pat DNA Gene Seq. 2010 Jan. 2 [Epub ahead of print]. “Nanoparticles in Gene Therapy Principles, Prospects, and Challenges”. Prog Mol Biol Transl Sci. 2011;104:509-62.

The 25HC3S compositions (preparations) may be administered by any of the many suitable means which are known to those of skill in the art, including but not limited to by injection (e.g. either systemically or via targeted injection, for example, into or into the vicinity of the liver), intravenously, inhalation, orally, intravaginally, intranasally, by ingestion of a food product containing the active agent, topically, by direct application to liver tissue, etc. In some aspects, the mode of administration is by injection or intravenously. In addition, the compositions may be administered in conjunction with other treatment modalities such as substances that lower serum lipids, agents that promote sensitivity to insulin, and the like.

Subjects to whom the compositions of the present disclosure are administered are generally mammals, and may be humans. However, the subject may also be a non-human mammal, e.g. a companion pet, or other non-human animal that could benefit from the therapy.

Also provided are methods of treating leptin-deficiency associated lipid accumulation in a subject in need thereof comprising:

selecting a subject having leptin-deficiency associated lipid accumulation; and

treating the subject with an effective amount of OCS or a pharmaceutically acceptable salt thereof.

In some embodiments, the subject having leptin-deficiency associated lipid accumulation is selected by a method comprising: measuring a value of one or more indicia of leptin deficiency in a subject; comparing the value with at least one negative control value or negative control range for the one or more indicia from a control population of individuals who are not leptin deficient; and if the value differs from that of the negative control value or falls outside the negative control range, then concluding that the subject has leptin deficiency associated lipid accumulation.

In some aspects, the methods of the present disclosure include identifying a subject that is in need of the treatments described herein, e.g. a subject who has leptin deficiency (LD), leptin resistance (LR), or a lipid storage disease (LSD) and thus may be predisposed to develop leptin-deficiency associated lipid accumulation (LDLA), leptin-resistance associated lipid accumulation (LRLA), lipid storage disease associated lipid accumulation (LSDLA) or who has or exhibits one or more indicia or signs associated with LD, LR, LDLA, LRLA, or a LSD, or is at risk of developing LD, LR, LDLA, LRLA, or a LSD, or a disease or condition associated with or caused by LD, LR, LDLA, LRLA or a LSD. Such a subject may be identified e.g. by measuring an indicium such as the amount of serum leptin and comparing the level to predetermined levels (ranges) measured in a suitable population of healthy control subjects and/or control subjects who are known to have LD, LR, and/or to have LDLA, LRLA, or a lipid storage disorder and/or control subjects being treated for one of these indications, and/or one or more control levels measured previously in the subject. For example, generally levels of leptin greater than about 10 ng/ml of serum, and usually greater than about 20 ng/ml, and more usually greater than about 25 ng/ml in a patient who still exhibits a deficiency in leptin levels, are interpreted as consistent with or indicating or diagnostic of leptin deficiency. Abnormal values for fasting blood sugar tests, AC1 tests (average of blood sugar levels measured during three months), PAI-1 (Plasminogen Activator Inhibitor-1), CRP tests (C-reactive Protein), urine tests, TSH (thyroid hormone) tests, measuring cholesterol levels, androgen levels, ultrasound checking of liver for fat deposition, etc., can be used to diagnose leptin resistance. The detection of fat globules (e.g. deposits of intracellular fat inside cell types other than adipocytes, such as a vacuole or droplet of triglyceride or some other lipid) is also an indicator of disease. Diagnosis of lipid storage disorders generally involves a demonstration of specific enzymatic deficiency in peripheral blood leukocytes or cultured fibroblasts, and a genetic analysis may be also be used. In addition, observable symptoms such as difficulty with muscle control, etc. may be the initial indicator of disease.

In other aspects, the methods of the present disclosure include identifying a subject that is in need of the treatments described herein, e.g. a subject who has abnormal lipid accumulation e.g. due to inadequate leptin activity (LD, LR, etc.) or a lipid storage disorder.

Diagnosis can be made through clinical examination, biopsy, genetic testing (for mutations predictive of the disorder), molecular analysis of cells or tissues, and enzyme assays (testing a variety of cells or body fluids for enzyme deficiency). A subject suffering from attenuated leptin activity or lipid storage disorder can further be selected based on the symptoms presented. For example, a subject diagnosed as suffering from Gaucher disease may based on the presence of an enlarged spleen and liver, liver malfunction, skeletal disorders and bone lesions that may cause pain and fractures, severe neurologic complications, swelling of lymph nodes and (occasionally) adjacent joints, distended abdomen, a brownish tint to the skin, anemia, low blood platelets, and yellow spots in the eyes, and/or the presence of a deficiency of the enzyme glucocerebrosidase.

Biopsy for lipid storage diseases involves removing, from a subject, a small sample of the liver or other tissue and studying it under a microscope, or subjecting the tissue sample to quantitative/qualitative assays. In this procedure, a physician administers a local anesthetic and then removes a small piece of tissue either surgically or by needle biopsy (a small piece of tissue is removed by inserting a thin, hollow needle through the skin). The tissue sample is analyzed for e.g. lipid deposition, or enzymatic activity. In addition, or optionally, genetic testing of the subject for lipid/glycogen storage disease may be done to determine if the subject is carrying a mutated gene that causes the disorder.

The beneficial effects exerted by administration of the active agents described herein include a decrease in one or more symptoms of LD, LR, LDLA, LRLA, or a lipid storage disorder, the decrease either occurring at a faster rate or to a more profound degree than would occur in the absence of OCS administration. Exemplary beneficial effects include but are not limited to a decrease in lipid levels e.g. in serum, in cells (such as liver cells), etc. of the subject and/or a decrease in fat in the liver, e.g. a decrease of at least about 10, 15, 20, 25, 30, 35, 40, 45, 50, 55, 60, 65, 70, 75, 80, 85, 90, 95 or even 100%, compared to a suitable control, e.g. a matched control subject or subjects to whom the active agents described herein have not been administered. Weight loss and/or weight stabilization may also occur.

In some aspects, the methods also include monitoring the effect of OCS administration in the subject. Accordingly, after a regimen of OCS administration has been started, one or more symptoms of the diseases/disorders discussed herein, may be observed, tracked, measured or monitored and a level of the extent, presence or development of the symptom is noted and compared to previous (pre-treatment, or earlier during treatment) levels. In addition, one or more indicia of LD, or LR, or a lipid storage disorder are monitored in this manner. For instance, in some cases, administration of an OCS reduces lipid accumulation in cells, tissue or organs e.g. of the liver, thereby reducing the size and/or number of lipid deposits that were present prior to treatment; and/or preventing further increases in the size and/or number of lipid deposits; reduces serum cholesterol; reduces plasma triglycerides; and/or reduces free fatty acids. Upon evaluation of the results, a skilled practitioner can evaluate the progress of the treatment and e.g. adjust the dose of OCS accordingly. For example, the dose is generally increased if symptoms are not being effectively treated, or decreased (or discontinued) if symptoms have been eradicated or lessened to a predetermined desired or targeted level, etc. For patients with a genetic predisposition to a disease which involves unwanted, abnormal and harmful lipid accumulation, treatment may be ongoing throughout their lifetime.

Subjects who will benefit from administration of an OCS such as 25HC3S are those who are experiencing, or are likely to experience, a symptom or condition associated with LD, LR, or a lipid storage disease “Associated with” refers to unwanted health-impairing symptoms, for example. Those that typically occur in individuals diagnosed with LD, LR, LDLA, LRLA or a lipid storage disorder. In some aspects, the symptoms are caused by defective leptin activity or a lipid storage disorder, may occur in conjunction with defective leptin activity or a lipid storage disorder, or may even cause defective leptin activity or a lipid storage disorder. In some aspects, the associated condition is LD- or LR-associated hyperlipidemia. “LD- or LR associated hyperlipidemia” means the condition of abnormally elevated levels of any or all lipids and/or lipoproteins in the blood of a subject with LD or LR. In other aspects, the associated condition is a lipid storage disease (LSD)-associated abnormal lipid deposition, e.g. in central or peripheral nervous tissue. Lipids and lipid composites that may be elevated in a subject and lowered by the treatments described herein include but are not limited to chylomicrons, very low-density lipoproteins, intermediate-density lipoproteins, low-density lipoproteins (LDLs), high-density lipoproteins (HDLs) and sphingolipids, as well as deposits or globules of these. In particular, elevated cholesterol (hypercholesterolemia) and triglycerides (hypertriglyceridemia) are known to be risk factors for blood vessel and cardiovascular disease due to their influence on atherosclerosis. Lipid elevation may also predispose a subject to other conditions such as acute pancreatitis. The methods of the present disclosure thus may also be used in the treatment or prophylaxis of conditions that are or are associated with elevated lipids, that include, for example, but are not limited to hyperlipidemia, hypercholesterolemia, hypertriglyceridemia, fatty liver (hepatic steatosis), and metabolic syndrome cardiovascular diseases, coronary heart disease, atherosclerosis, acute pancreatitis, various metabolic disorders, such as insulin resistance syndrome, diabetes, polycystic ovary syndrome, fatty liver disease, cachexia, obesity, atherosclerosis, arteriosclerosis, stroke, gall stones, inflammatory bowel disease, and the like. In addition, various conditions associated with hyperlipidemia include those described in issued U.S. Pat. No. 8,003,795 (Liu, et al) and U.S. Pat. No. 8,044,243 (Sharma, et al), the complete contents of both of which are herein incorporated by reference in their entireties.

Generally, “prophylactic” or “prophylaxis” relates to a reduction in the likelihood of the patient developing a disorder or symptom associated with LD, such as LDLA, or associated with LR, such as LRLA, or associated with a lipid storage disease. For example, the methods can be used prophylactically as a measure designed to preserve health and prevent the spread or maturation of disease in a patient. It is also appreciated that the various modes of treatment or prevention of a disease can mean “substantial” treatment or prevention, which includes total but also less than total treatment or prevention, and in which some biologically or medically relevant result is achieved, e.g., amelioration and/or managing of disease symptoms, improvement in quality of life, etc. Furthermore, treatment or treating as well as alleviating can refer to therapeutic treatment and prophylactic or preventative measures in which the object is to prevent, or to slow (lessen) progress of a disease state, condition or malady. For example, a treatment is successful if, after administration of an effective or therapeutic amount of 25HC3S, the treated subject shows observable and/or measurable reduction in or absence of one or more signs and symptoms of an LD-associated disease or condition, an LR-associated disease or condition, or a lipid storage disorder and/or reduced morbidity and mortality, and/or improvement in quality of life.

In one aspect, the subject that is treated is an individual having a lack of leptin activity due to one or more genetic abnormalities. For example, individuals with one or more mutations in the Ob(lep) gene that encodes the leptin receptor, or a mutation in the LEP gene encoding leptin, may be treated by the methods described herein. The leptin receptor is present in the hypothalamus and individuals with mutations in the receptor, especially those who are homozygous for a mutation, generally suffer from obesity and other severe life-impairing and life-threatening abnormalities beginning very early in life. Associated symptoms include chronic excessive eating (hyperphagia); obesity; abnormal eating behaviors (e.g. fighting with other children over food, hoarding food, eating in secret, etc.); hypogonadotropic hypogonadism (reduced production of hormones that direct sexual development, which may delay or prevent puberty and may cause infertility); increased susceptibility to Entamoeba histolytica infections; and others. Individuals with mutations in the LEP gene may exhibit similar symptoms.

In order to treat, prevent or reverse these symptoms, in one aspect, a subject who is treated using the methods described herein is a child (even a very young child such as a newborn or infant) who has tested positive for a mutation in the gene encoding leptin or the leptin receptor and/or who exhibits one or more additional symptoms of a deficiency in leptin activity . A test for leptin receptor mutations may be carried out at or prior to birth, e.g. in utero via amniocentesis or other suitable technique, or post-partum using known genetic testing methods. Alternatively, genetic testing of one or both parents, the results of which show that the fetus/infant has a high probability of having a leptin or leptin receptor mutation with or without testing of the infant/fetus per se, may also indicate the advisability of administering an OCS such as 25HC3S. Administration of an OCS can begin immediately e.g. after birth, or even prior to birth. For example, an OCS may be administered to a pregnant female whose fetus has been diagnosed as carrying a leptin receptor mutation, so that the fetus is exposed to or contacted with the agent during gestation. Alternatively, or in addition, an OCS may be administered to a nursing mother of such an individual to elevate levels of the OCS in breast milk that is ingested by the infant, or by some other suitable means.

Herein, “fetus” refers to an unborn offspring of a mammal; “neonate” refers to a newborn child or offspring, especially one that is less than one month old; “infant” refers to a human child between the ages of 1 month and 2 years of age; “child” refers to a human subject below the age of puberty (onset of overt sexual development); “adolescent” refers to a human subject from puberty to adulthood; “adult” refers to a sexually mature individual fully capable of reproduction (or who would be capable of reproduction but for an abnormality or physical condition preventing the same such as genetically induced LD, LR or a LSD), e.g. for a human, at least about 13 years of age. Those of skill in the art will recognize that the same or similar terms may apply to non-human species treated by the methods described herein.

In another aspect, the subject that is treated is an individual who suffers from a congenital lipid storage disorder. Such disorders are typically characterized by the unwanted or abnormal buildup (accumulation) of lipids in cells or tissues. Lipid storage disorders include, for example, neutral lipid storage disease, Gaucher disease, Niemann-Pick disease, Fabry disease, Farber's disease, gangliosidoses such as GM1 gangliosidoses and GM2 gangliosidoses (e.g. Tay -Sachs disease and Sandhoff disease), Krabbe disease, metachromatic leukodystrophy (MLD, including late infantile, juvenile, and adult MLD), and acid lipase deficiency disorders such as Wolman's disease and cholesteryl ester storage disease. Each of these diseases is caused by at least one genetic mutation that results in a non-functional or poorly functional enzyme that is involved in lipid storage. For example: Gaucher's disease, a lysosomal storage disease, results from a deficiency of the enzyme glucocerebrosidase (glucosylceramidase), and is characterized by the buildup of sphingolipids cells and organs.

Farber disease, a lysosomal storage disease, deficiency in the enzyme ceramidase that causes an accumulation of fatty material lipids leading to abnormalities in the joints, liver, throat, tissues and central nervous system.

Nieman-Pick disease is a lysosomal storage disease in which the fat, sphingomyelin, accumulates in lysosomes. Types A and B are caused by mutations in the gene encoding the enzyme acid sphingomyelinase. Type C is caused by a mutation in a gene encoding a transport protein of the endosomal-lysosomal system

Wolman's disease is due to a mutation is the gene encoding lysosomal acid lipase (LAL or LIPA) and is a lysosomal storage disorder. A late onset form is known as cholesteryl ester storage disease

Metachromatic leukodystrophy, a lysosomal storage disease, is caused by a defect in the gene encoding arylsulfatase A, and causes accumulation of fats called sulfatides in cells.

Fabry disease, a lysosomal storage disease, is caused by a mutation in the gene encoding alpha-galactosidase A and leads to accumulatation of globotriaosylceramide (Gb3, GL-3, or ceramide trihexoside) within blood vessels, other tissues, and organs.

Neutral lipid storage disease myopathy subtype (NLSD-M) is caused by loss-of-function mutations in PNPLA2, which encodes adipose triglyceride lipase (ATGL, also known as desnutrin).

Gangliosidosis is a sub-category of Sphingolipidosis that contains two different types of lipid storage disorders caused by the accumulation of lipids known as gangliosides. The GM I gangliosidoses are caused by a deficiency of beta-galactosidase, with resulting abnormal storage of acidic lipid materials in cells of the central and peripheral nervous systems, but particularly in the nerve cells. GM2 gangliosidoses are caused by a deficiency of the enzyme beta-hexosaminidase which catalyzes the biodegradation of fatty acid derivatives known as gangliosides. For example, Tay-Sachs disease is caused by mutations in the HEXA gene (encoding the alpha-subunit of beta-N-acetylhexosaminidase A, a lysosomal enzyme) which cause abnormalities in the gene product.

In one embodiment the lipid storage disorder is Neutral lipid storage disease (NLSD) (also known as “Chanarin-Dorfman syndrome”). NLSD is an autosomal recessive disorder in which lipids are stored abnormally in organs and tissues throughout the body characterized by accumulation of triglycerides in the cytoplasm of leukocytes, muscle, liver, fibroblasts, and other tissue. Individuals with NLSD suffer from cardiac and skeletal myopathy and hepatic steatosis due to the accumulation of fats in muscle tissue. Other features of NLSD include fatty liver, a weakened and enlarged heart (cardiomyopathy), inflammation of the pancreas (pancreatitis), and reduced thyroid activity (hypothyroidism). Mutations in adipocyte triglyceride lipase (ATGL) regulatory protein CGI-58 cause NLSD-I, with additional symptoms of neurological defects (Lefevre et al. Am. J. Hum. Genet. 2001). In one embodiment, the lipid storage disorder is neutral lipid storage disease with myopathy (NLSD-M). Mutations in the PNPLA2 gene cause NLSD-M, which is characterized by more severe myopathy than NLSD. The PNPLA2 gene encodes the enzyme adipose triglyceride lipase (ATGL), which plays a role in breaking triglycerides, the main source of stored energy in cells. PNPLA2 gene mutations impair the ATGL enzyme's ability to break down triglycerides, which then accumulate in muscle and tissues throughout the body, resulting in the signs and symptoms of neutral lipid storage disease with myopathy. Two out of six patients identified with NLSD-M die from heart failure (Fischer et al. Nat. Genet. 2007).

In one embodiment the lipid storage disorder is a Niemann-Pick disease. Niemann-Pick disease is a group of autosomal recessive disorders caused by an accumulation of fat and cholesterol in cells of the liver, spleen, bone marrow, lungs, and, in some patients, brain. Neurological complications may include ataxia, eye paralysis, brain degeneration, learning problems, spasticity, feeding and swallowing difficulties, slurred speech, loss of muscle tone, hypersensitivity to touch, and some corneal clouding. A characteristic cherry-red halo develops around the center of the retina in 50 percent of patients.

In one embodiment the lipid storage disorder is Gaucher disease. Gaucher disease is the most common of the lipid storage diseases and is caused by a deficiency of the enzyme glucocerebrosidase. Fatty material often collects in the spleen, liver, kidneys, lungs, brain, and bone marrow. Symptoms may include enlarged spleen and liver, liver malfunction, skeletal disorders and bone lesions that may cause pain and fractures, severe neurologic complications, swelling of lymph nodes and (occasionally) adjacent joints, distended abdomen, a brownish tint to the skin, anemia, low blood platelets, and yellow spots in the eyes. Persons affected most seriously may also be more susceptible to infection. The disease affects males and females equally.

Gaucher disease has three common clinical subtypes. Type I (or normeuropathic type) is the most common form of the disease. It occurs most often among persons of Ashkenazi Jewish heritage.

Symptoms of Gaucher disease may begin early in life or in adulthood and include enlarged liver and grossly enlarged spleen, which can rupture and cause additional complications. Skeletal weakness and bone disease may be extensive. The brain is not affected, but there may be lung and, rarely, kidney impairment. Patients in this group usually bruise easily due to low blood platelets and experience fatigue due to anemia. Depending on disease onset and severity, type 1 patients may live well into adulthood. Many patients have a mild form of the disease or may not show any symptoms. Type 2 (or acute infantile neuropathic Gaucher disease) typically begins within 3 months of birth. Symptoms include an enlarged liver and spleen, abnormal eye movement, extensive and progressive brain damage, spasticity, seizures, limb rigidity, and a poor ability to suck and swallow. Affected children usually die before age 2. Type 3 (the chronic neuronopathic form) can begin at any time in childhood or even in adulthood. It is characterized by slowly progressive but milder neurologic symptoms compared to the acute or Type 2 Gaucher disease. Major symptoms include an enlarged spleen and/or liver, seizures, poor coordination, skeletal irregularities, eye movement disorders, blood disorders including anemia, and respiratory problems. Patients often live to their early teen years and, in some cases, into adulthood.

In some embodiments, the compounds of the present disclosure are administered in conjunction with additional therapies known to be effective in the disorder. For example, for type 1 and most type 3 Gaucher disease patients, enzyme replacement treatment can be given intravenously every two weeks and can dramatically decrease liver and spleen size, reduce skeletal abnormalities, and reverse other manifestations. Surgery to remove the spleen may be required on rare occasions (if the patient is anemic or when the enlarged organ affects the patient's comfort). Blood transfusion may benefit some anemic patients. Other patients may require joint replacement surgery to improve mobility and quality of life.

Niemann-Pick disease is currently subdivided into four categories. Onset of type A, the most severe form, is in early infancy. Infants appear normal at birth but develop an enlarged liver and spleen, swollen lymph nodes, nodes under the skin (xanthemas), and profound brain damage by 6 months of age. The spleen may enlarge to as much as 10 times its normal size and can rupture. These children become progressively weaker, lose motor function, may become anemic, and are susceptible to recurring infection. They rarely live beyond 18 months. This form of the disease occurs most often in Jewish families. In the second group, called type B (or juvenile onset), enlargement of the liver and spleen characteristically occurs in the pre-teen years. Most patients also develop ataxia, peripheral neuropathy, and pulmonary difficulties that progress with age, but the brain is generally not affected. Type B patients may live a comparatively long time but many require supplemental oxygen because of lung involvement. Niemann-Pick types A and B result from accumulation of the fatty substance called sphingomyelin, due to deficiency of an enzyme called sphingomyelinase.

Niemann-Pick disease also includes two other variant forms called types C and D. These may appear early in life or develop in the teen or even adult years. Niemann-Pick disease types C and D are not caused by a deficiency of sphlingomyelinase but by a lack of the NPC1 or NPC2 proteins. As a result, various lipids and particularly cholesterol accumulate inside nerve cells and cause them to malfunction. Patients with types C and D have only moderate enlargement of their spleens and livers. Brain involvement may be extensive, leading to inability to look up and down, difficulty in walking and swallowing, and progressive loss of vision and hearing. Type D patients typically develop neurologic symptoms later than those with type C and have a progressively slower rate of loss of nerve function. Most type D patients share a common ancestral background in Nova Scotia. The life expectancies of patients with types C and D vary considerably. Some patients die in childhood while others who appear to be less severely affected can live into adulthood. Children usually die from infection or progressive neurological loss.

In some embodiments of the present disclosure, the compounds of the present disclosure are administered in conjunction with additional therapies, e.g. patients with types C and D are frequently placed on a low-cholesterol diet and/or cholesterol lowering drugs, although research has not shown these interventions change the abnormal cholesterol metabolism or halt progression of the disease.

In one embodiment the lipid storage disorder is Fabry disease. Fabry disease, also known as alpha-galactosidase-A deficiency, causes a buildup of fatty material in the autonomic nervous system, eyes, kidneys, and cardiovascular system. Fabry disease is the only X-linked lipid storage disease. Males are primarily affected although a milder form is common in females. Occasionally, affected females have severe manifestations similar to those seen in males with the disorder. Onset of symptoms is usually during childhood or adolescence. Neurological signs include burning pain in the arms and legs, which worsens in hot weather or following exercise, and the buildup of excess material in the clear layers of the cornea (resulting in clouding but no change in vision). Fatty storage in blood vessel walls may impair circulation, putting the patient at risk for stroke or heart attack. Other manifestations include heart enlargement, progressive kidney impairment leading to renal failure, gastrointestinal difficulties, decreased sweating, and fever. Angiokeratomas (small, non-cancerous, reddish-purple elevated spots on the skin) may develop on the lower part of the trunk of the body and become more numerous with age. Patients with Fabry disease often die prematurely of complications from heart disease, renal failure, or stroke.

In some embodiments, the compounds are administered in conjunction with additional therapies, e.g. drugs such as phenyloin and carbamazepine are often prescribed to treat pain that accompanies Fabry disease and metoclopramaide or Lipisorb (a nutritional supplement) can ease gastrointestinal distress that often occurs in Fabry patients, and some individuals may require kidney transplant or dialysis. Enzyme replacement can reduce storage, ease pain, and improve organ function in patients with Fabry disease.

Farber's disease, also known as Farber's lipogranulomatosis, describes a group of rare autosomal recessive disorders that cause an accumulation of fatty material in the joints, tissues, and central nervous system. The disorder affects both males and females. Disease onset is typically in early infancy but may occur later in life. Children who have the classic form of Farber's disease develop neurological symptoms within the first few weeks of life. These symptoms may include moderately impaired mental ability and problems with swallowing. The liver, heart, and kidneys may also be affected. Other symptoms may include vomiting, arthritis, swollen lymph nodes, swollen joints, joint contractures (chronic shortening of muscles or tendons around joints), hoarseness, and xanthemas which thicken around joints as the disease progresses. Patients with breathing difficulty may require insertion of a breathing tube. Most children with the disease die by age 2, usually from lung disease. In one of the most severe forms of the disease, an enlarged liver and spleen (hepatosplenomegaly) can be diagnosed soon after birth. Children born with this form of the disease usually die within 6 months.

Farber's disease is caused by a deficiency of the enzyme called ceramidase. Currently there is no specific treatment for Farber's disease. Corticosteroids may be prescribed to relieve pain. Bone marrow transplants may improve granulomas (small masses of inflamed tissue) on patients with little or no lung or nervous system complications. Older patients may have granulomas surgically reduced or removed.

In one embodiment the lipid storage disorder is a gangliosidose. The gangliosidoses are comprised of two distinct groups of genetic diseases. Both are autosomal recessive and affect males and females equally. The GMI gangliosidoses are caused by a deficiency of the enzyme beta-galactosidase, resulting in abnormal storage of acidic lipid materials particularly in the nerve cells in the central and peripheral nervous systems. GMI gangliosidosis has three clinical presentations: early infantile, late infantile, and adult. Signs of early infantile GM1 (the most severe subtype, with onset shortly after birth) may include neurodegeneration, seizures, liver and spleen enlargement, coarsening of facial features, skeletal irregularities, joint stiffness, distended abdomen, muscle weakness, exaggerated startle response, and problems with gait. About half of affected patients develop cherry-red spots in the eye. Children may be deaf and blind by age 1 and often die by age 3 from cardiac complications or pneumonia. Onset of late infantile GM1 gangliosidosis is typically between ages 1 and 3 years. Neurological signs include ataxia, seizures, dementia, and difficulties with speech. Onset of adult GM1 gangliosidosis is between ages 3 and 30. Symptoms include muscle atrophy, neurological complications that are less severe and progress at a slower rate than in other forms of the disorder, corneal clouding in some patients, and dystonia (sustained muscle contractions that cause twisting and repetitive movements or abnormal postures). Angiokeratomas may develop on the lower part of the trunk of the body. The size of the liver and spleen in most patients is normal.

The GM2 gangliosidoses also cause the body to store excess acidic fatty materials in tissues and cells, most notably in nerve cells. These disorders result from a deficiency of the enzyme beta-hexosaminidase. The GM2 disorders include Tay-Sachs disease and Sandhoff diseases.

Tay-Sachs disease (also known as GM2 gangliosidosis-variant B). Tay-Sachs and its variant forms are caused by a deficiency in the enzyme hexosaminidase A. The incidence is particularly high among Eastern European and Ashkenazi Jewish populations, as well as certain French Canadians and Louisianan Cajuns. Affected children appear to develop normally for the first few months of life. Symptoms begin by 6 months of age and include progressive loss of mental ability, dementia, decreased eye contact, increased startle reflex to noise, progressive loss of hearing leading to deafness, difficulty in swallowing, blindness, cherry-red spots in the retinas, and some paralysis. Seizures may begin in the child's second year. Children may eventually need a feeding tube and they often die by age 4 from recurring infection. No specific treatment is available. Anticonvulsant medications may initially control seizures. Other supportive treatment includes proper nutrition and hydration and techniques to keep the airway open. A rarer form of the disorder, called late-onset Tay-Sachs disease, occurs in patients in their twenties and early thirties and is characterized by unsteadiness of gait and progressive neurological deterioration.

Sandhoff disease (variant AB). This is a severe form of Tay-Sachs disease. Onset usually occurs at the age of 6 months and is not limited to any ethnic group. Neurological signs may include progressive deterioration of the central nervous system, motor weakness, early blindness, marked startle response to sound, spasticity, myoclonus (shock-like contractions of a muscle), seizures, macrocephaly (an abnormally enlarged head), and cherry-red spots in the eye. Other symptoms may include frequent respiratory infections, murmurs of the heart, doll-like facial features, and an enlarged liver and spleen. There is no specific treatment for Sandhoff disease. As with Tay-Sachs disease, supportive treatment includes keeping the airway open and proper nutrition and hydration. Anticonvulsant medications may initially control seizures. Children generally die by age 3 from respiratory infections.

In one embodiment the lipid storage disorder is Krabbe disease. Krabbe disease (also known as globoid cell leukodystrophy and galactosylceramide lipidosis) is an autosomal recessive disorder caused by deficiency of the enzyme galactocerebrosidase. The disease most often affects infants, with onset before age 6 months, but can occur in adolescence or adulthood. The buildup of undigested fats affects the growth of the nerve's protective myelin sheath and causes severe deterioration of mental and motor skills. Other symptoms include muscle weakness, hypertonia (reduced ability of a muscle to stretch), myoclonic seizures (sudden, shock-like contractions of the limbs), spasticity, irritability, unexplained fever, deafness, optic atrophy and blindness, paralysis, and difficulty when swallowing. Prolonged weight loss may also occur. The disease may be diagnosed by its characteristic grouping of cells into globoid bodies in the white matter of the brain, demyelination of nerves and degeneration, and destruction of brain cells. In infants, the disease is generally fatal before age 2. Patients with a later onset form of the disease have a milder course of the disease and live significantly longer.

In one embodiment the lipid storage disease is a Metachromatic leukodystrophy (MLD). MLD is a group of disorders marked by storage buildup in the white matter of the central nervous system and in the peripheral nerves and to some extent in the kidneys. Similar to Krabbe disease, MLD affects the myelin that covers and protects the nerves. This autosomal recessive disorder is caused by a deficiency of the enzyme arylsulfatase A. Both males and females are affected by this disorder.

MLD has three characteristic phenotypes: late infantile, juvenile, and adult. The most common form of the disease is late infantile, with onset typically between 12 and 20 months following birth. Infants may appear normal at first but develop difficulty in walking and a tendency to fall, followed by intermittent pain in the arms and legs, progressive loss of vision leading to blindness, developmental delays, impaired swallowing, convulsions, and dementia before age 2. Children also develop gradual muscle wasting and weakness and eventually lose the ability to walk. Most children with this form of the disorder die by age 5. Symptoms of the juvenile form typically begin between ages 3 and 10. Symptoms include impaired school performance, mental deterioration, ataxia, seizures, and dementia. Symptoms are progressive with death occurring 10 to 20 years following onset. In the adult form, symptoms begin after age 16 and may include impaired concentration, depression, psychiatric disturbances, ataxia, seizures, tremor, and dementia. Death generally occurs within 6 to 14 years after onset of symptoms.

Bone marrow transplantation may delay progression of the disease in some cases. Considerable progress has been made with regard to gene therapies in animal models of MLD.

In one embodiment the lipid storage disorder is Wolman's disease. Wolman's disease, also known as acid lipase deficiency, is a severe lipid storage disorder that is usually fatal by age 1. This autosomal recessive disorder is marked by accumulation of cholesteryl esters (normally a transport form of cholesterol) and triglycerides (a chemical form in which fats exist in the body) that can build up significantly and cause damage in the cells and tissues. Both males and females are affected by this disorder. Infants are normal and active at birth but quickly develop progressive mental deterioration, enlarged liver and grossly enlarged spleen, distended abdomen, gastrointestinal problems including steatorrhea (excessive amounts of fats in the stools), jaundice, anemia, vomiting, and calcium deposits in the adrenal glands, causing them to harden.

In one embodiment the lipid storage disorder is Cholesteryl ester storage disease, which is characterized by an acid lipase deficiency. Cholesteryl ester storage disease results from storage of cholesteryl esters and triglycerides in cells in the blood and lymph and lymphoid tissue. Children develop an enlarged liver leading to cirrhosis and chronic liver failure before adulthood. Children may also have calcium deposits in the adrenal glands and may develop jaundice late in the disorder.

Treatment of subjects with lipid storage disorders preferably begins as early as possible, and is generally carried out and monitored as described above for other diseases characterized by abnormal lipid accumulation. Treatment may also be provided in conjunction with enzyme replacement therapy.

In some aspects, the populations of subjects treated by the methods described herein may or may not have symptoms of and/or been diagnosed with one or more of high levels of cholesterol (hypercholesterolemia, e.g. cholesterol levels in serum in the range of about 200 mg/dl or more), or with a condition associated with high levels of cholesterol e.g. hyperlipidemia, atherosclerosis, heart disease, stroke, Alzheimer's, gallstone diseases, cholestatic liver diseases, etc. In some aspects, the populations of subjects treated by the methods described herein may or may not have symptoms of and/or been diagnosed with hypertriglyceridemia, or with a condition associated with hypertriglyceridemia. In some aspects, the populations of subjects treated by the methods described herein do not have symptoms of and/or have not been diagnosed with high levels of cholesterol (hypercholesterolemia, e.g. cholesterol levels in serum in the range of about 200 mg/dl or more), or with a condition associated with high levels of cholesterol e.g. hyperlipidemia, atherosclerosis, heart disease, stroke, Alzheimer's, gallstone diseases, cholestatic liver diseases, etc.

In further aspects, the populations of subjects treated by the methods described herein may or may not have symptoms of and/or been diagnosed with liver disorders such as hepatitis, inflammation of the liver, caused mainly by various viruses but also by some poisons (e.g. alcohol); autoimmunity (autoimmune hepatitis) or hereditary conditions; non-alcoholic fatty liver disease, a spectrum in disease, associated with obesity and characterized by an abundance of fat in the liver, which may lead to hepatitis, i.e. steatohepatitis and/or cirrhosis; cirrhosis, i.e. the formation of fibrous scar tissue in the liver due to replacing dead liver cells (the death of liver cells can be caused, e.g. by viral hepatitis, alcoholism or contact with other liver-toxic chemicals); haemochromatosis, a hereditary disease causing the accumulation of iron in the body, eventually leading to liver damage; cancer of the liver (e.g. primary hepatocellular carcinoma or cholangiocarcinoma and metastatic cancers, usually from other parts of the gastrointestinal tract); Wilson's disease, a hereditary disease which causes the body to retain copper; primary sclerosing cholangitis, an inflammatory disease of the bile duct, likely autoimmune in nature; primary biliary cirrhosis, an autoimmune disease of small bile ducts; Budd-Chiari syndrome (obstruction of the hepatic vein); Gilbert's syndrome, a genetic disorder of bilirubin metabolism, found in about 5% of the population; glycogen storage disease type II; as well as various pediatric liver diseases, e.g. including biliary atresia, alpha-I antitrypsin deficiency, alagille syndrome, and progressive familial intrahepatic cholestasis, etc. In addition, liver damage from trauma may also be treated, e.g. damage caused by accidents, gunshot wounds, etc. Further, liver damage caused by certain medications may be prevented or treated, for example, drugs such as the antiarrhythmic agent amiodarone, various antiviral drugs (e.g. nucleoside analogues), aspirin (rarely as part of Reye's syndrome in children), corticosteroids, methotrexate, tamoxifen, tetracycline, etc. are known to cause liver damage. In further aspects, the populations of subjects treated by the methods described herein do not have symptoms of and/or have not been diagnosed with liver disorders such as hepatitis, inflammation of the liver, caused mainly by various viruses but also by some poisons (e.g. alcohol); autoimmunity (autoimmune hepatitis) or hereditary conditions; non-alcoholic fatty liver disease, a spectrum in disease, associated with obesity and characterized by an abundance of fat in the liver, which may lead to hepatitis, i.e. steatohepatitis and/or cirrhosis; cirrhosis, i.e. the formation of fibrous scar tissue in the liver due to replacing dead liver cells (the death of liver cells can be caused, e.g. by viral hepatitis, alcoholism or contact with other liver-toxic chemicals); haemochromatosis, a hereditary disease causing the accumulation of iron in the body, eventually leading to liver damage; cancer of the liver (e.g. primary hepatocellular carcinoma or cholangiocarcinoma and metastatic cancers, usually from other parts of the gastrointestinal tract); Wilson's disease, a hereditary disease which causes the body to retain copper; primary sclerosing cholangitis, an inflammatory disease of the bile duct, likely autoimmune in nature; primary biliary cirrhosis, an autoimmune disease of small bile ducts; Budd-Chiari syndrome (obstruction of the hepatic vein); Gilbert's syndrome, a genetic disorder of bilirubin metabolism, found in about 5% of the population; glycogen storage disease type II; as well as various pediatric liver diseases, e.g. including biliary atresia, alpha-1 antitrypsin deficiency, alagille syndrome, and progressive familial intrahepatic cholestasis, etc. In addition, liver damage from trauma may also be treated, e.g. damage caused by accidents, gunshot wounds, etc. Further, liver damage caused by certain medications may be prevented or treated, for example, drugs such as the antiarrhythmic agent amiodarone, various antiviral drugs (e.g. nucleoside analogues), aspirin (rarely as part of Reye's syndrome in children), corticosteroids, methotrexate, tamoxifen, tetracycline, etc. are known to cause liver damage.

In further aspects, the populations of subjects treated by the methods described herein may or may not have symptoms of non-alcoholic fatty liver disease (NAFLD) and/or nonalcoholic steatohepatitis (NASH). In further aspects, the populations of subjects treated by the methods described herein do not have symptoms of non-alcoholic fatty liver disease (NAFLD) and/or nonalcoholic steatohepatitis (NASH).

In further aspects, the populations of subjects treated by the methods described herein may or may not have symptoms of one or more diseases and conditions which lead to and/or cause, or are otherwise associated with: organ or organ system dysfunction/failure, e.g. dysfunction or failure of one or more organs or organ systems such as the liver, kidney, heart, brain, pancreas, cardiovascular, respiratory, renal, haematological, neurological, gastrointestinal organs, hepatic organs, lungs, intestines, colon, and spleen, including Multiple Organ Dysfunction Syndrome (MODS) and/or acute liver failure and/or kidney failure and organ dysfunction or failure caused by acetaminophen (ATMP); ischemia such as cardiac, brain, bowel, limb, and cutaneous ischemia; inflammation, tissue necrosis, organ necrosis, stroke, and reperfusion injury; or organ dysfunction/failure associated with cardiovascular surgery, heart surgery, aneurysm surgery, liver surgery and transplant surgery. In further aspects, the populations of subjects treated by the methods described herein do not have symptoms of and/or have not been diagnosed with one or more diseases and conditions which lead to and/or cause, or are otherwise associated with organ or organ system dysfunction/failure, e.g. dysfunction or failure of one or more organs or organ systems such as the liver, kidney, heart, brain, pancreas, cardiovascular, respiratory, renal, haematological, neurological, gastrointestinal organs, hepatic organs, lungs, intestines, colon, and spleen, including Multiple Organ Dysfunction Syndrome (MODS) and/or acute liver failure and/or kidney failure and organ dysfunction or failure caused by acetaminophen (ATMP); ischemia such as cardiac, brain, bowel, limb, and cutaneous ischemia; inflammation, tissue necrosis, organ necrosis, stroke, and reperfusion injury; or organ dysfunction/failure associated with cardiovascular surgery, heart surgery, aneurysm surgery, liver surgery and transplant surgery.

In further aspects, administration of an OCS to a subject as described herein may or may not coincidentally, as a result of administration, treat another secondary condition such as hyperlipidemia, and/or may have a beneficial activity such as decreasing intracellular lipids, treating lipid accumulation in NAFLD, decreasing inflammation, inhibiting cholesterol biosynthesis, decreasing fat accumulation in liver cells, promoting liver proliferation or liver tissue regeneration, inhibiting lipid biosynthesis, reducing cholesterol, reducing inflammation, or treating e.g. diabetes, hyperlipidemia, atherosclerosis, fatty liver disease and/or inflammatory disease. However, the methods described herein are intended primarily to treat disease symptoms related to attenuated leptin activity and/or lipid storage disorders in a subject in need thereof, which have been diagnosed as described herein, whether or not the subject has other disorders, or symptoms of other disorders, whether or not the “other disorders” have been formally diagnosed.

As used herein, the transitional phrase “consisting essentially of” (and grammatical variants) is to be interpreted as encompassing the recited materials or steps and those that do not materially affect the basic and novel characteristic(s) of the claimed invention. See MPEP 2111.03. Thus, the term “consisting essentially of” as used herein is not equivalent to “comprising.”

The present invention will be further illustrated by way of the following Example. This Example is non-limiting and does not restrict the scope of the invention. Unless stated otherwise, all percentages, parts, etc. presented in the examples are by weight.

### EXAMPLE

In vivo Effects of 25HC3S Administration on Symptoms and Conditions Associated with Leptin Resistance

### Methods

### Animal Studies

To examine the effect of 25HC3S on lipid accumulation in sera and in the liver in a leptin-deficient (obese fa/fa) animal model (Zucker rats), 11 week old Zucker rats (Charles River, Wilmington, Mass.) were randomly assigned into four groups. Obese fa/fa Zucker rats have a defective leptin receptor and serve as a general animal model for deficiencies in leptin activity, whether due to low leptin levels, congenital defects in the leptin receptor or acquired leptin resistance, since all result in inadequate or faulty leptin activity. Further, these animals develop unwanted lipid accumulation after several weeks of life due to a genetic mutation in the leptin receptor. All rats were housed under identical conditions with a 12-hour light/12-hour dark cycle and given free access to water and food. The rats (n=5 in each treatment) were gavaged orally with vehicle (10% cellulose) or 25HC3S (10 mg/kg, 30 mg/kg, and 100 mg/kg) once a day for 21 days and fasted overnight (14 hrs) before collection of samples. To compare with gavage administration, other rats (n=10-17 in each treatment) were intraperitoneally injected with vehicle solution (ethanol/PBS; vehicle) or 25HC3S (50 mg/kg) once every three days for 6 weeks and fasted overnight (14 hrs). Blood samples were collected before sacrifice. Serum triglyceride (TG), total cholesterol (CHOL), high density lipoprotein-cholesterol (HDL-C), alkaline phosphatase (ALK), alanine aminotransferase (ALT), and aspartate aminotransferase (AST) were measured using standard enzymatic techniques. Lipoprotein profiles in sera were analyzed by HPLC in the following descriptions.

### HPLC analysis of Serum Lipoprotein Profiles

The lipoproteins, triglycerides and cholesterol in very low density lipoprotein (VLDL), low-density lipoprotein (LDL) and high-density lipoprotein (HDL) were measured by gel filtration using HPLC as previously described (Bai et al., 2012). Briefly, rat serum (100 μl) was eluted with a solvent of 154 mM NaCl with 0.1 mM EDTA (pH 8.0) at a flow rate of 0.2 ml/min via a Pharmacia Superose 6 HR 10/30 FPLC column, and the signal was read at a wavelength of 280 nm. Fractions were collected for 1.2 min each from 20 min up to 100 min. 180 μl of each fraction plus 20 μl of a 10× solution of Wako Total Cholesterol E Reagent (Wako Chemicals USA, Richmond, VA) was incubated in a 96-well plate at 37° C. for 3 hrs in the dark to analyze cholesterol levels, and the absorbance was read at a wavelength of 595 nm. 2 μl of each fraction plus 200 p1 of Infinity™ Triglycerides Reagent (Fisher Scientific, Pittsburgh, Pa.) was incubated in a 96-well plate at 37° C. for 5 minutes in dark to analyze triglyceride levels, and the absorbance was read at a wavelength of 500 nm. The lipoprotein profile was monitored as an internal control.

### Histo-Morphology Analysis

Three specimens from different regions of the liver of each rat were collected and fixed in 4% paraformaldehyde in 0.1 M phosphate buffer at room temperature overnight. The regions of the specimens were standardized for all rats. The paraffin-embedded tissue sections (4 μm) were stained with hematoxylin and eosin (HE). 3-3′-Diaminobenzidine (DAB) was used as the chromogen, and hematoxylin was used as the nuclear counterstain. Determination of total intracellular lipids: Oil Red O staining of intracellular neutral lipids: the liver slides were fixed with 3.7% formaldehyde in PBS for 30 min followed by two washes with phosphate-buffered saline (PBS). The cells were stained with 0.2% Oil Red O in 60% 2-propanol for 10 min and washed three times with PBS. The images were taken with the use of a microscope (Olympus, Tokyo, Japan) equipped with image recorder under 40× lenses.

All slides of tissue were qualitatively examined by light microscopy by a board certified veterinary pathologist. The incidence and severity of the lesions were scored. Lesions were also assessed for duration and distribution.

### Quantification of Hepatic Lipids

Liver tissues were homogenized, and the hepatic total lipids were extracted using a mixture of chloroform and methanol (2:1, v:v). After the extracts were filtered, 0.2 ml of each was evaporated to dryness and dissolved in 100 μl of isopropanol containing 10% Triton™ X-100 for cholesterol assay (Wako Chemicals USA, Richmond, Va.), 100 μl of NEFA solution (0.5 g of EDTA-Na_{z}, 2 g of Triton™ X-100, 0.76 ml of 1N NaOH, and 0.5 g of sodium azide/l, pH 6.5) for the free fatty acid assay (Wako Chemicals USA, Richmond, Va.), or 100 μl of isopropanol only for the triglyceride assay (Fisher Scientific, Pittsburgh, Pa.). Each lipid concentration was normalized by liver weight.

### Statistical Analysis

All results were expressed as means±standard errors. Statistical analysis was performed with Student t-test for unpaired samples. A value of p <0.05 was considered as statistically significant.

### Results

In vivo treatment with 25HC3S prevented or reversed LR symptoms in subjects with the disease. In particular, administration of 25HC3S resulted in decreased serum lipid levels and decreased deposition of fat in the liver of the subjects.

### Effects of 25HC3S Administration on Lipid Homeostasis in Zucker Rats

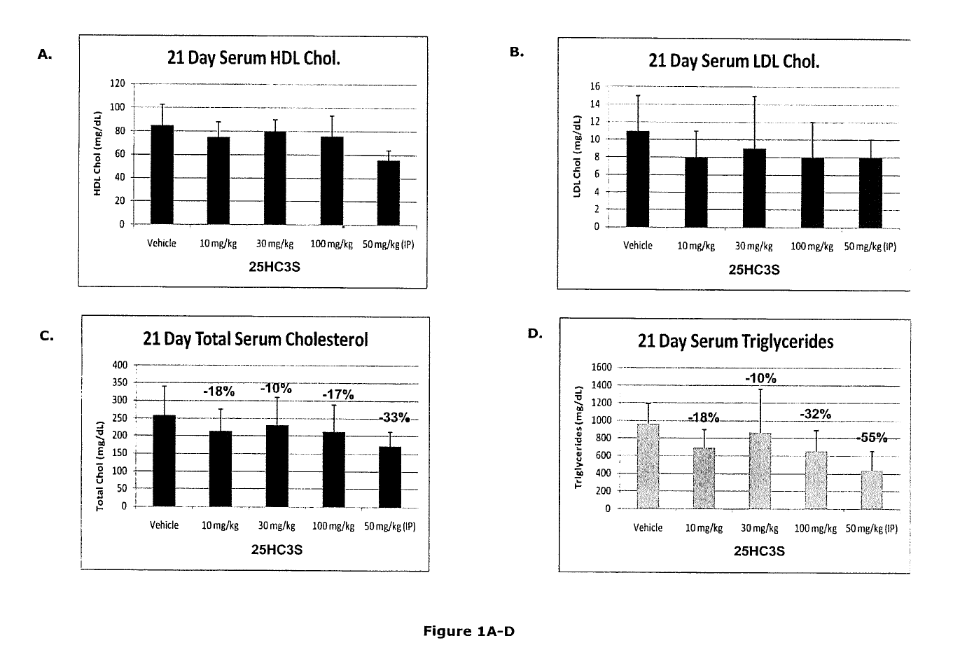

25HC3S has been shown to reduce lipid accumulation in both primary hepatocytes and THP-1 macrophages (Ma et al., 2008; Ren et al., 2007; Xu et al., 2010). To investigate the effects of 25HC3S administration on hyperlipidemia in leptin-resistance Zucker rats, the rats were treated with 25HC3S or vehicle for 21 days and fasted overnight. The treatments with gavage administration of 25HC3S significantly decreased plasma TG and CHOL, by 30% and 17% respectively as shown in FIG. 1 while no significant change in HDL (FIG. 1A) and LDL levels (FIG. 1B). Interestingly, intraperitoneal administration significantly decreased total cholesterol and triglyceride as well as HDL (FIGS. 1A, C and D), and more efficiency than gavage administration. The results indicate that 25HC3S decreases these lipid levels not only in liver tissue but also in peripheral tissues.

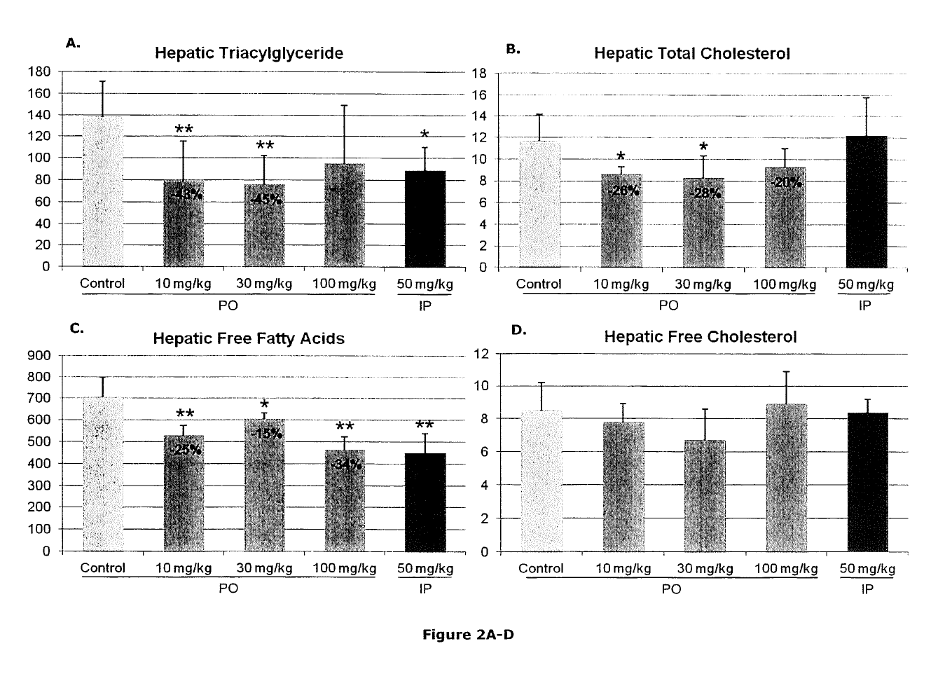

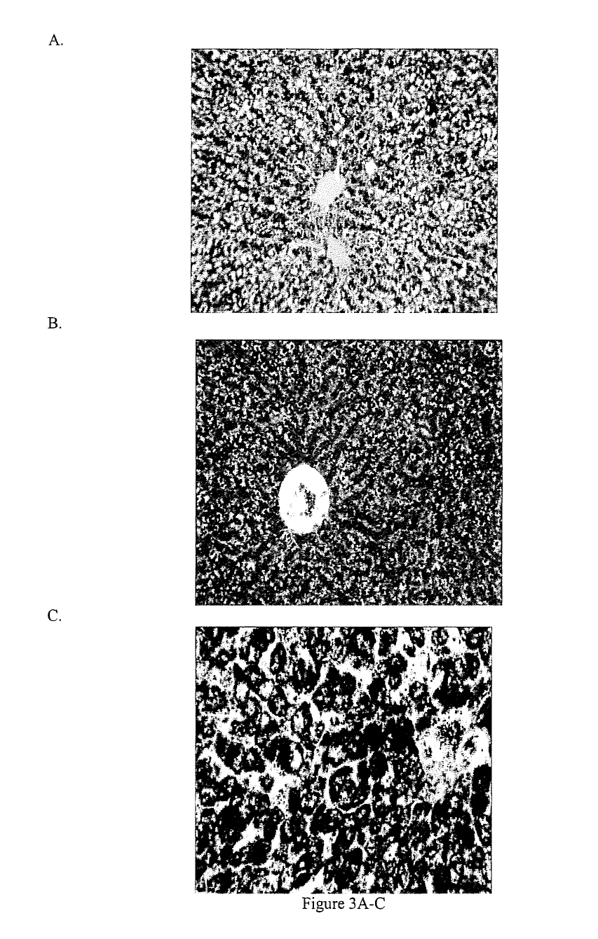

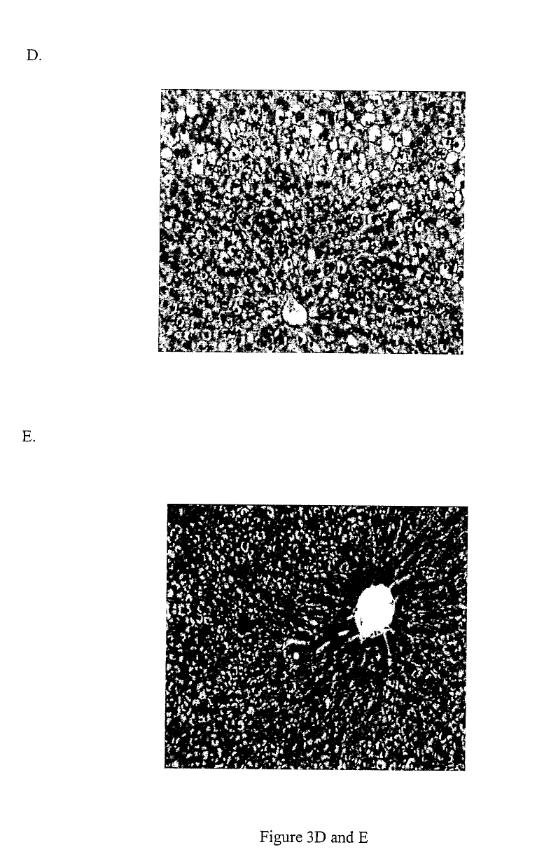

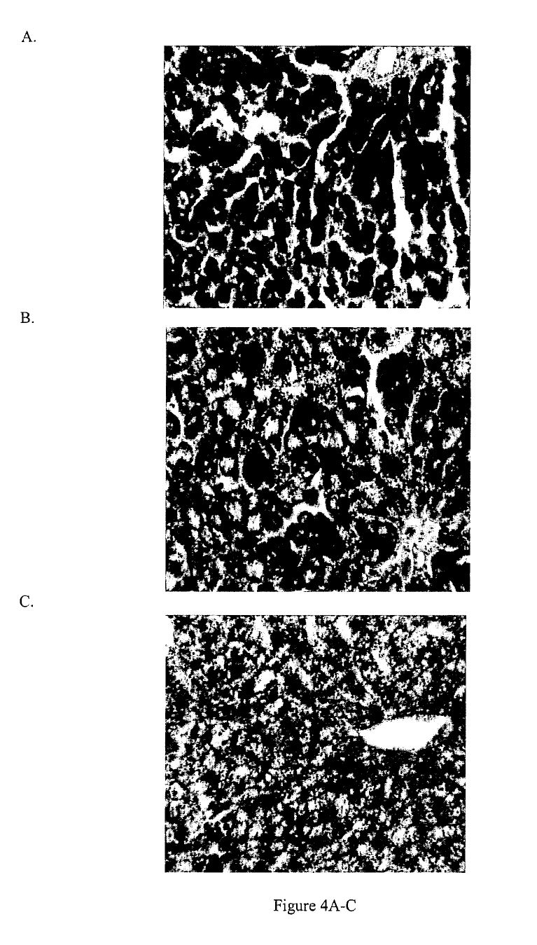

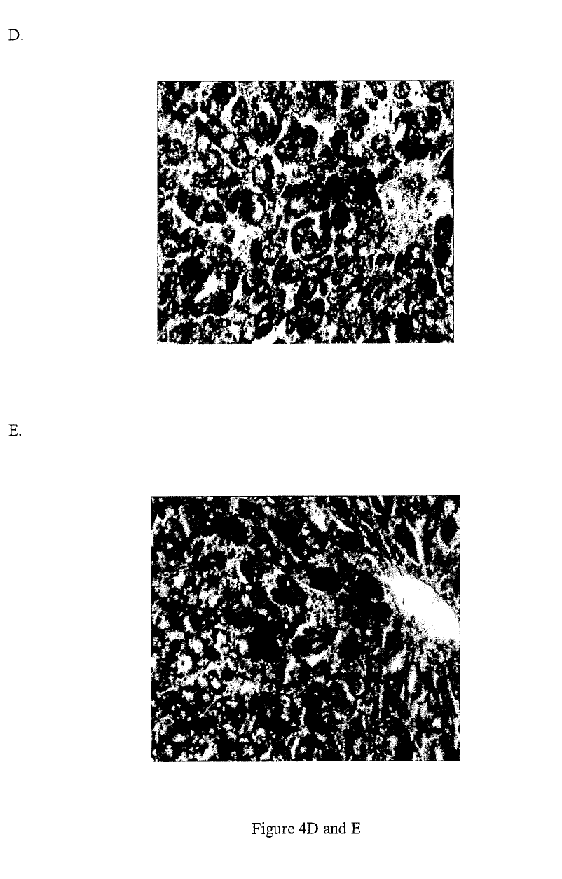

To study the effect of 25HC3S on hepatic lipid metabolism, lipid levels were measured in liver tissues. The results showed that gavage administration of 10 mg/kg of 25HC3S significantly reduced lipids in the liver tissues, e.g., triglyceride, total cholesterol, and free fatty acids levels by 43%, 25%, and 26%, respectively (FIGS. 2A, B, and C). It was noticed that the free cholesterol concentration was not affected by the administration of 25HC3S (FIGS. 2D). The decreases in lipid levels were further confirmed by morphological analysis (FIGS. 3 and 4). The liver tissues of control rats were pale and were distended by large cytoplasmic lipid inclusions (FIGS. 3A), suggesting that the leptin deficiency model was successfully established. However, the administration of 25HC3S significantly decreased lipid inclusions in hepatocytes (FIGS. 3B, C, and D). The lipid accumulation was confirmed by Oil Red O staining as shown in FIG. 4. The results are summarized as shown in Table 1.

TABLE 1
Histologic findings of hepatic lipidosis

| | | 25HC3S | | |
|---|---|---|---|---|
| | | (mg/kg/day, | | |
| | | except as | | Force |
| Group | | indicated | Severity of | Rank** |
| No. * | Animal No. | for IP) | Hepatic Lipidosis | (Oil Red O) |
| 1 | 1001-1005 | 0 | Moderate to severe | 5 |
| 2 | 2001-2005 | 10 | Mild to moderate | 4 |
| 3 | 3001-3005 | 30 | Mild to moderate | 3 |
| 4 | 4001-4005 | 50 | Mild | 2 |
| | | (IP every | | |
| | | 3 days) | | |
| 5 | 5001-5005 | 100 | Mild | 1 |

* N = 5 males
**1 = least severe, 5 = most severe

Thus, this study shows for the first time that when 25HC3S is administered to leptin resistant subjects in vivo, symptoms and conditions associated with LR (e.g., elevated serum lipids and lipid accumulation in liver) are effectively treated or prevented.

### References

Bai, Q., Zhang, X., Xu, L., Kakiyama, G., Heuman, D M., Sanyal, A., Pandak, W M., and Ren, S. Oxysterol sulfation by overexpression of hydroxysteroid sulfotransferase SULT2B1b suppresses SREBP-1c signaling and reduces hepatic lipids in mouse models of nonalcoholic fatty liver diseases. Metabolism: Clinical and Experimental, 61, 836-845, 2012.
Ma, Y., Xu, L., Rodriguez-Agudo, D., Li, X., Heuman, D., Hylemon, P., Pandak, W M., and Ren, S. 25-Hydroxycholesterol-3-Sulfate (25HC3S) regulates macrophage lipid metabolism via the LXR/SREBP-1 Signaling Pathway. AJP-Endocrinology and Metabolism, 295, E1369, 2008.
Ren, S., Li, X., Rodriguez-Agudo, D., Gil, G., Hylemon, P., and Pandak, WM. Sulfated Oxysterol, 25HC3S, is a Potent Regulator of Lipid Metabolism in Human Hepatocytes. Biochem. Biophysic. Res. Commun., 6, 802, 2007.
Xu, L., Bai, Q., Rodriguez-Agudo, D., Hylemon, P., Heuman, D M., Pandak, W M., and Ren, S. Regulation of hepatocyte lipid metabolism and inflammatory response by 25-hydroxycholesterol and 25-Hydroxycholesterol-3-sulfate, Lipids, 45, 821-832, 2010.
Zhang, Z., Li, D., Blanchard, D. E., Lear S. R., Erickson, S. K., and Spencer T. A. Key regulatory oxysterols in liver: analysis as 44-3-ketone derivatives by HPLC and response to physiological perturbations. J Lipid Res. 42, 649-658, 2001.

Unless otherwise stated, a reference to a compound or component includes the compound or component by itself, as well as in combination with other compounds or components, such as mixtures of compounds.

As used herein, the singular forms “a,” “an,” and “the” include the plural reference unless the context clearly dictates otherwise.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

1. A method of treating leptin deficiency in a subject in need thereof,
the method comprising elevating in the subject an amount of at least one oxygenated cholesterol sulfate (OCS), or a pharmaceutically acceptable salt thereof, wherein the elevating is sufficient to treat or alleviate symptoms of leptin deficiency, and wherein the elevating comprises administering the at least one OCS or the pharmaceutically acceptable salt thereof to the subject.

2. The method of claim 1, wherein the at least one OCS is selected from 5-cholesten-3, 25-diol, 3-sulfate (25HC3S); 5-cholesten, 3b, 25-diol, disulfate (25HCDS); (5-cholestene, 3, 27-diol, 3-sulfate); (5-cholestene, 3, 27-diol, 3, 27-disulfate); (5-cholestene, 3,7-diol, 3-sulfate); (5-cholestene, 3,7-diol, 3,7-disulfate); (5-cholestene, 3, 24-diol, 3-sulfate); (5-cholestene, 3, 24-diol, 3, 24-disulfate); and (5-cholestene, 3-ol, 24, 25-epoxy 3-sulfate).

3. The method of claim 1, wherein the elevating is sufficient to reduce lipids and/or lipid deposits in one or more of the circulatory system, blood or internal organs of the subject.

4. (canceled)

5. The method of claim 1, wherein the at least one OCS or a pharmaceutically acceptable salt thereof is administered in an amount ranging from 0.1 mg/kg to 100 mg/kg, based on the body mass of the subject.

6. The method of claim 5, wherein the at least one OCS or a pharmaceutically acceptable salt thereof is administered in an amount ranging from 10 mg/kg to 30 mg/kg, based on the body mass of the subject.

7. The method of claim 1, wherein the administering comprises at least one of oral administration, enteric administration, sublingual administration, transdermal administration, intravenous administration, peritoneal administration, parenteral administration, administration by injection, subcutaneous injection, and intramuscular injection.

8.-20. (canceled)
